(19) United States
(12) Patent Application Publication
(10) Pub. No.: US 2020/0325449 A1
(43) Pub. Date: Oct. 15, 2020

(54) COMPLEMENT INHIBITION FOR IMPROVING CELL VIABILITY

(71) Applicant: THE CLEVELAND CLINIC FOUNDATION, Cleveland, OH (US)

(72) Inventor: Feng Lin, Cleveland, OH (US)

(21) Appl. No.: 16/305,911

(22) PCT Filed: Jun. 2, 2017
(86) PCT No.: PCT/US17/35652

§ 371 Date: Nov. 30, 2018

Related U.S. Application Data
(60) Provisional application No. 62/344,460, filed on Jun. 2, 2016.

(51) Int. Cl. C12N 5/0775; A61K 35/28; A61L 33/00

(52) U.S. Cl. CPC C12N 5/0663; C12N 2500/34; A61L 33/0011; A61K 35/28

(57) ABSTRACT

Methods of increasing immunogenic cell viability by administering an immunogenic animal cell and a complement inhibitor to a subject are described. Immunogenic animal cells such as stem cells protected with a coating of complement inhibitor are also described.

### CONTINUING APPLICATION DATA

This application claims the benefit of U.S. Provisional Application Ser. No. 62/344,460 filed Jun. 2, 2016, which is incorporated by reference herein.

### GOVERNMENT FUNDING

This invention was made with government support under Grant Nos. DK103581 and AR061564, awarded by the National Institutes of Health. The Government has certain rights in the invention.

### BACKGROUND

Mesenchymal stem cells (MSCs) are adult stem cells that not only have the ability to differentiate into different type of cells, including osteoblasts, adipocytes, and chondrocytes, but also possess strong immunosuppressive activity on both the innate and adaptive immune system through multiple mechanisms. Importantly, it is widely believed that MSCs are able to escape host immune surveillance because of this immunosuppressive activity and other features. Tsuji et al., Circ Res 2010, 106(10): 1613-1623. It has therefore been assumed that MSCs expanded from one donor could be used to treat other patients without being rejected. Because of the convenience of preparation, timing, cost effectiveness, and lack of need for HLA matching, many clinical trials on MSCs have used allo-MSCs. Ankrum J, Karp J M., Trends Mol Med 2010, 16(5): 203-209. Infusion of MSCs by intravenous (i.v.) injection is the most common delivery route in both humans and animals. Infusion of allo- or xeno-MSCs has been found to be effective in ameliorating pathological conditions in many animal models of disease, including MS6, rheumatoid arthritis, myasthenia gravis, diabetes, IBD10, and allograft rejection, thus providing the rationale for testing the use of MSCs in clinical trials. However, in both human and animal studies, it has been observed that most of the infused cells are first trapped in the lung, then some migrate out to other tissues and mysteriously disappear within a few days. Ankrum J, Karp J M., Trends Mol Med 2010, 16(5): 203-209. The extremely low survival rate of administered MSCs suggests that they work in a “hit and run” fashion, and therefore the initial survival and health state of the MSCs after administration are critical for their therapeutic efficacy. von Bahr et al., Stem cells 2012, 30(7): 1575-1578.

Complement is an important part of the innate immune system, the primary role of which is to serve as the first defense against foreign pathogens. Activated complement can directly attack invading pathogens by forming membrane attack complexes (MACs) to damage/lyse the foreign cells. To avoid pathogenic bystander attack on self-tissues, complement activation is tightly controlled by native complement regulators, both in the fluid phase and on self-cell surfaces. However, when the naturally existing complement regulatory mechanisms fail to control excessive activation of complement, this leads to tissue damage and diseases. Because of the important pathological roles of complement in many diseases, pharmaceutical companies are developing complement inhibitors as potential therapeutics. One of these, a humanized anti-C5 monoclonal antibody (mAb), is in clinical use for treating paroxysmal nocturnal hemoglobinuria, atypical hemolytic-uremic syndrome, and myasthenia gravis. Nester C M, Brophy P D., Curr Opin Pediatr 2013, 25(2): 225-231.

The inventors recently reported that, immediately after infusion, MSCs activate complement in the blood and are injured by MACs, leading to reduced viability and decreased functionality, a process involving naturally existing antibodies in the blood. Li Y, Lin F., Blood 2012, 120(17): 3436-3443. The inventors also demonstrated that either systemic inhibition of complement using anti-C5 antibodies or transfection of MSCs with a recombinant adenovirus to upregulate levels of a cell surface complement inhibitor, CD55, significantly reduces serum-mediated MSC damage. These studies provide proof-of-concept that either systemic inhibition of complement in the blood or local inhibition of complement activation on the MSC surface is effective in protecting MSCs. However, transfecting MSCs with a recombinant virus is costly and has many safety concerns for clinical use, whereas long-term systemic inhibition of complement by administration of anti-C5 mAb has practical issues, including cost (current anti-C5 mAb therapy costs $400,000/year/patient) and side-effects (e.g. opportunistic infections). It is therefore clear that a new, economical, safe, and effective approach needs to be developed.

### SUMMARY

The results of recent clinical trials using mesenchymal stem cells (MSCs) have been unsatisfactory, showing that current MSC-based therapies need to be improved. The inventors and others have previously demonstrated that MSCs activate complement by unknown mechanisms after infusion, leading to damaged MSCs. In this report, the inventors found that incorporation of N-glycolylneuraminic acid onto MSCs during in vitro culture was a factor in the activation of complement by MSCs. In addition, the inventors developed a method to “paint” heparin onto MSCs, which improved MSC viability and enhanced their function after infusion by directly inhibiting complement and by recruiting factor H, another potent complement inhibitor in the serum, onto MSCs.

In addition, the inventors have developed a simple method for painting factor H, a native complement inhibitor, onto MSCs to locally inhibit complement activation on MSCs. MSCs painted with factor H are protected from both MAC- and neutrophil-mediated attack and are significantly more effective in inhibiting antigen-specific T cell responses than the mock-painted MSCs both in vitro and in vivo. These data suggest that cell surface engineering of MSCs with a complement inhibitor such as factor H or heparin to locally inhibit complement activation on MSCs might be a useful method for improving the outcome of current MSC-based therapies.

### BRIEF DESCRIPTIONS OF THE DRAWINGS

FIGS. 1A-1C provide graphs showing sialic acid Neu5GC on in vitro propagated MSCs triggers complement-mediated damage. A. MSCs from different donors (640, 678, 741, 742, and 784) were evaluated for the presence of Neu5GC by flow cytometry after staining with anti-Neu5GC IgY (solid lines) or control IgY (dotted lines). B. left panel: Levels of Neu5GC on MSCs cultured for 7 days in the absence or presence of Neu5AC. Dotted line and solid line: MSCs incubated with Neu5AC and stained with control IgY (IgG) (dotted line) or with anti-Neu5GC IgY (IgG) (solid line). Dashed lines and shaded areas, MSCs cultured in the absence of Neu5AC and stained with control IgY(IgG) (dashed line) or anti-Neu5GC IgY (IgG) (shaded area). middle panels: Levels of IgM and IgGs on these MSCs after incubation with 30% serum for 30 min. Right panel, levels of C3b deposition on MSCs cultured in the absence or presence of Neu5AC after incubating them with 30% serum for 30 min in GVB++. C. Complement-mediated cell damage when MSCs cultured in the presence (black bars) or absence (gray bars) of Neu5AC were incubated with 20% or 30% normal human serum in GVB++. The data are the combined results for 3 individual experiments±SEM, *p<0.05.

FIGS. 2A and 2B provide graphs showing systemic administration of heparin inhibits complement activation and protects MSCs from complement-mediated damage. A. Ex vivo evaluation of complement activation in heparin-injected or control mice. Mice were injected s.c. with heparin (500 U/mouse) or the same volume of saline and plasma was collected 30 min later and incubated with antibody-sensitized sheep erythrocytes (E^{shA}) in GVB++(classical pathway) or zymosan in GVB EGTA Mg++(alternative pathway) in the presence or absence of 1 mM EDTA, then C3b/iC3b on the surface was evaluated after FITC-labeled anti-mouse C3 IgG staining followed by flow cytometric analyses. Dotted lines, EDTA-treated samples; solid lines, samples from saline-treated mice; shades areas, samples from heparin-treated mice. B. In vivo MSC damage after infusion into heparin- or saline-injected mice. Mice were injected s.c. with heparin (500 U/mouse) or saline, then, 10 min later, BCECF-labeled MSCs (1×10⁶/mouse) were infused via the tail vein and in vivo MSC damage at different time points assessed by measuring released BCECF in the blood. n=2 in each group, * p<0.05.

FIGS. 3A-3E provide graphs showing Heparin can be painted onto MSCs to locally inhibit complement and protect the cells. (A and B) MSCs were incubated with different concentrations of FITC-labeled non-activated heparin (A) or activated heparin (B), then, after washing, MSC-bound heparin was assessed by flow cytometry. C. MSCs painted with 100 μg/ml of heparin or mock-painted MSCs were incubated with 30% normal human serum in GVB++ for 30 min at 37° C., then were washed and stained with FITC-labeled anti-human C3 IgGs to evaluate local complement activation (C3b/iC3b deposition). D. MSCs painted with different concentrations of activated heparin were labeled with BCECF, then incubated with either 20% normal human serum (gray bars) or 30% normal human serum (black bars) in GVB++ for 30 min at 37° C., and cell damage was assessed by the BCECF release assay. The data are the combined results from 3 independent experiments±SEM. *p<0.05. E. Heparin-painted or mock-painted MSCs (0.5×10⁶/mouse) were labeled with BCECF and infused into WT C57BL/6 mice via the tail vein, then in vivo MSC damage was assessed by measuring released BCECF in the blood at different time points. n=6 in each group, *p<0.05

FIGS. 4A and 4B provide graphs showing painted heparin recruits complement factor H (CfH) onto MSCs and the heparin-recruited CfH is integrally involved in protecting MSCs from complement-mediated attack. A. Heparin-painted MSCs were incubated with either 30% factor H-depleted human serum (solid line) or 30% normal human serum (shaded area) in GVB-EDTA buffer for 30 min. After washing, MSC surface-bund CfH were evaluated by flow cytometry after staining with a goat anti-human CfH IgG. Representative results from 2 independent experiments. B. Heparin-painted MSCs were incubated with 30% normal human serum (black bar), 30% CfH-depleted serum (light gray bar), or 30% CfH-depleted serum supplemented with 200 μg/ml of purified CfH in GVB-EDTA for 30 min (EDTA was used in this step to allow factor H recruitment without the occurrence of complement activation). After washes, the cells were labeled with BCECF, then incubated with 30% CfH-depleted serum in GVB++ for another 30 min and cell damage was assessed by BCECF release (Factor H-depleted serum had to be used in this step as the source of complement to avoid the interference of factor H presented in the normal serum). The data are the combined results of 2 independent experiments±SEM, *p<0.05.

FIGS. 5A and 5B provide graphs showing painted heparin does not interfere with the T cell inhibitory activity of MSCs before contact with serum and preserves MSC function after contact with serum. A. Heparin-painted MSCs (Hep-MSCs) or mock-painted MSCs (Ctrl-MSCs) (both treated with mytomycin C to prevent proliferation) were incubated for 30 min at 37° C. with 30% normal human serum in GVB++ or GVB++ alone. After washes, they were then co-cultured with 4×10⁵ anti-CD3/CD28-activated T cells at different ratios for 72 h, then the proliferation of the activated T cells was measured by BrdU incorporation. B. Culture supernatants from these co-cultures were assayed for IFNγ released by the activated T cells by IFNγ ELISA. The data are the combined results for 2 independent experiments±SEM, *p<0.05

FIGS. 6A-6D provide graphs showing heparin-painted MSCs (Hep-MSCs) are more effective in inhibiting the proliferation of antigen-specific T cells in vivo than mock-painted MSCs (Ctrl-MSCs). Naïve C57BL/6 mice were infused with 1×10⁶ purified T cells from OT-II mice via the tail vein, then, 24 h later, OVA-loaded dendritic cells (DCs) were injected into the hind footpads. The mice were then randomly divided into 2 groups, one of which was injected with 1×10⁶ heparin-painted MSCs and the other with the same number of mock-painted MSCs via the tail vein. After 24 h, 1 mg of BrdU was injected i.p. into each mouse, then, 24 h later, the mice were euthanized and the draining lymph nodes collected to assess the proliferation of activated T cells by BrdU incorporation using a conventional BrdU ELISA. A-D are the results from 4 independent experiments with a total of 14 mice in each group. Each dot represents one mouse±SD, *p<0.05

FIGS. 7A-7D provide graphs and images showing heparin-painted MSCs (Hep-MSCs) survive better in vivo than mock-painted MSCs (Ctrl-MSCs). Using the same experimental setup as above, one side of the draining (popliteal) lymph nodes were collected, and the percentage of live MSCs evaluated by staining the single cell suspension with an anti-human HLA-ABC IgG, followed by flow cytometric analysis (A & B); the distal non-draining (cervical) lymph nodes were collected and processed at the same time as controls (C & D). n=5 in each group, *p<0.05.

FIGS. 8A-8C provide graphs showing neutrophils are activated by complement after MSC infusion. MSCs were infused into WT or C3 KO mice (1×10⁶/mouse) by tail vein injection, then, after 40 min, peripheral blood was collected and activation of neutrophils in the blood assessed by staining the cells with DHR123, followed by flow cytometric analysis. WT mice without MSC infusion were included as controls. (A) Neutrophils in the peripheral blood. The oval gate indicates the cells analyzed in B and C. (B) Representative results showing activated neutrophils (DHR123+) in WT mice after MSC infusion (WT w/MSCs), C3 KO mice after MSC infusion (C3 KO w/MSCs), and control WT mice without MSC infusion (WT w/o MSCs). (C) Combined results for neutrophil activation assessment after MSC infusion. n=5 in each group and show the mean±SD, One-way ANOVA and Tukey post-hoc test were used for data analysis, *p<0.05

FIGS. 9A-9D provide graphs showing MSCs are damaged by complement-activated neutrophils after administration. MSCs (1×10⁶) were labeled with BCECF-AM and infused into WT mice depleted of neutrophils using anti-NIMP Ab (WT NIMP w/MSCs), WT controls not depleted of neutrophils (WT Ctrl IgG w/MSCs), and C3 KO mice (C3 KO w/MSCs). WT mice without MSC infusion (WT w/o MSCs) were included as controls. (A) At 0, 20, 40 and 60 min, blood was collected and levels of BCECF released from damaged MSCs were measured. Two-way ANOVA tests showed a significant difference between each group, * p<0.05. (B-D) Six hours after MSC infusion, the lung, liver, and spleen were collected and single cell suspensions prepared and incubated with Cy5.5-labeled anti-human CD105 mAb, a marker of human MSCs, followed by flow cytometric analysis. (B) Because most cells in the lymph nodes are small lymphocytes, large and living cells based on FSC/SSC profile which should contain infiltrated MSCs were gated and analyzed in C and D. (C and D) The large and living cells were then analyzed for the presence of surviving MSCs (BCECF+CD105+); (C) shows representative results for surviving MSCs and (D) shows the combined results for all mice studied. n=4 in each group. One-way ANOVA and Tukey post-hoc test were used for data analysis, * p<0.05.

FIG. 10 provides a graph showing activated neutrophils act synergistically with complement to damage MSCs. MSCs (2×10⁴) were labeled with BCECF and incubated for 30 min with 30% NHS alone (NHS) or 2×10⁴ HL60 cell-derived neutrophils alone (Neut) or in the presence of various concentrations of wortmannin (0, 0.001, 0.01, 0.1, and 1 μM) (Neut+Wort), or 30% NHS plus 2×10⁴ neutrophils alone (Neut+NHS) or in the presence of various concentrations of wortmannin (0, 0.001, 0.01, 0.1 and 1 M) (Neut+NHS+Wort), then MSC damage was quantified using the BCECF release assay. The data are representative results in triplicate for 3 independent experiments and show the mean±SD, One-way ANOVA and Tukey post-hoc tests were used for data analysis, *p<0.05.

FIGS. 11A and 11B provide graphs showing C5aR, but not C3aR, signaling activates neutrophils to injure the administrated MSCs. (A) MSCs (2×10⁴) were labeled with BCECF, then incubated for 30 min with different combinations of 30% NHS (NHS), HL60 cell-derived neutrophils (2×10⁴) (Neut.), a C3aR antagonist (C3aRA, 10 μM), and/or a C5aR antagonist (C5aRA, 10 μM), then MSC damage was quantified using the BCECF release assay. (B) MSCs (2×10⁴) were labeled with BCECF, then were incubated for 30 min with primary neutrophils (2×10⁴) purified from peripheral blood in the presence or absence of purified C5a (50 ng/ml), then MSC damage was assessed as above. The data are representative results in triplicate for 3 independent experiments mean±SD, One-way ANOVA and Tukey post-hoc tests were used for data analysis, *p<0.05

FIGS. 12A-12E provide graphs showing factor H can be painted onto MSCs to locally inhibit complement activation. (A) MSCs were incubated for 30 min with 0, 2.5, 5, 10, or 20 μg of EDC-activated CFH or 20 μg of non-activated CFH (20 C) in 100 μl of PBS for painting, then, after washes, CFH on MSCs was measured by incubation for 30 min at 4° C. with goat anti-CFH antibodies and for 30 min at 4° C. with Alexa 488-labeled donkey anti-goat IgG antibodies, followed by flow cytometric analysis. iso, isotype control for the anti-CFH IgGs on cells painted with 20 μg of EDC-activated CFH. The result shown is representative of those obtained in 5 independent experiments. (B) To evaluate how long the painted CFH can stay on MSCs, MSCs were painted with CFH (10 μg/ml), and cultured in wells of 6-well plates. Levels of painted CFH on the cell surface (mean fluorescence intensity) were quantitated by flow cytometric analysis every 12 hr for the first 36 hr, then daily for up to 5 days. (C) To assess local complement activation, MSCs were first incubated for 30 min with 0, 2.5, 5, or 10 μg of EDC-activated CFH or 10 μg of non-activated CFH (10 C) for painting, then, after washing, the cells were incubated with or without (Ctrl) 30% NHS in GVB++ for 30 min, then C3b/iC3b deposited on the cell surface were quantified by flow cytometry after staining the cells with a fluorescein isothiocyanate-conjugated anti human C3 IgG. The result shown is representative of those obtained in 3 independent experiments. (D and E) MSCs painted with 10 μg of EDC-activated CFH (CFH-MSC) or mock-painted (Crtl-MSC) were incubated for 30 min with 30% NHS, then the supernatants were collected and C3a (D) and C5a (E) measured using an anaphylatoxin cytometric bead array kit. The data are representative results in triplicate for 3 independent experiments and are the mean±SD, multiple t-test was used for data analysis, *p<0.05

FIGS. 13A and 13B provide graphs showing painting factor H onto MSCs protects MSCs from the synergistic effect of complement and neutrophils. (A) MSCs (2×10⁴) were incubated with 0, 2.5, 5, or 10 μg of EDC-activated CFH (Act. CFH) or 10 μg of non-activated CFH (Ctrl) for painting, then were labeled with BCECF, washed, then incubated with 30% NHS in GVB++ for 30 min and MSC damage was quantified using the BCECF leakage assay. Two-way ANOVA tests were used for data analysis, * p<0.05. (B) MSCs (2×10⁴) were painted with (CFH-MSC) or without (Ctrl-MSC) 10 μg of EDC-activated CFH, labeled with BCECF, and then were incubated for 30 min with 30% NHS alone (NHS), 2×10⁴ neutrophils alone (Neut.) or 30% NHS plus with 2×10⁴ neutrophils (NHS+Neut), then MSC damage was measured using the BCECF leakage assay. The data are representative of results in triplicate for 4 independent experiments and show the mean±SD, One-way ANOVA and Tukey post-hoc tests were used for data analysis, *p<0.05

FIGS. 14A and 14B provide graphs showing painting CFH onto MSCs preserves their viability and function after contact with complement in vitro. MSCs were painted with (CFH-MSCs) or without (Ctrl-MSCs) 10 μg of EDC-activated CFH, then were incubated for 30 min with 30% NHS in GVB++(NHS) or GVB++ alone (w/o NHS). After washing and irradiation, 2×10⁴ of the MSCs were cultured in each well of a 96 well plate with different numbers of PBMCs in the presence of anti-CD3/CD28 beads and 30 U/ml of IL-2. After 48 h, 10 M BrdU was added in each well, then, after 24 h, (A) the cells were harvested and proliferation of activated T cells was assessed by measuring levels of incorporated BrdU using a BrdU ELISA kit, and (B) supernatants were collected to measure IFNγ produced by the activated T cells. The data are results in triplicate representative of those in 4 independent experiments and are the mean±SD, Two-way ANOVA tests were used in data analysis, *p<0.05.

FIGS. 15A-15D provide graphs showing painting CFH onto MSCs improves their viability and function in vivo. (A) Naïve WT mice were injected with 5×10⁶ in vitro propagated IRBP₁₋₂₀-specific Th17 cells via the tail vein injection, then half received 0.5×10⁶ MSCs painted with 10 μg/ml of CFH (CFH-MSC) and the other half the same number of mock-painted MSCs (Ctrl MSCs). After 7 days, the spleens were collected and 1×10⁶ splenocytes from each mouse incubated for 72 h in the presence of 20 μg/ml of IRBP₁₋₂₀ (IRBP), 20 μg/ml of irrelevant OVA323-339 peptide (OVA), or no peptide (no Ag), then IRBP₁₋₂₀-specific Th17 responses were assessed by measuring IL-17 in the culture supernatants using conventional ELISA. The results are the mean±SD, n=4 in each group, One-way ANOVA and Tukey post-hoc tests were used for data analysis, *p<0.05 comparing the indicated groups. (B-D) Naïve WT mice were injected with 4×10⁶ CFSE-labeled OT II T cells via the tail vein, then, after 24 h, syngeneic bone marrow-derived DCs loaded with 1 μg/ml of OVA (0.4×10⁶/mouse) were injected s.c. into the hind footpads and CFH-painted MSCs (CFH-MSC) or mock-painted MSCs (Ctrl MSC) (1×10⁶/mouse) were injected via the tail vein. After another 24 h, 1 mg of BrdU was injected i.p. into each mouse, then, 24 h later, the draining lymph nodes (popliteal lymph nodes) were harvested and T cell proliferation quantified by measuring CFSE dilution and flow cytometry and by measuring incorporated BrdU using a BrdU ELISA. Representative T cell proliferation results based on CFSE-dilution measurements from Ctrl-MSC or CFH-MSC treated mice are presented in dot plot (B) and histogram (C). T cell proliferation assessments based on CFSE-dilution measurements (D) and BrdU incorporation (E) results from three different experiments were normalized against the average value from the Ctrl MSC-treated mice (as 100%) in each experiment and combined. n=6 in each group, mean±SEM, multiple t-test was used for data analysis, *p<0.05.

### DETAILED DESCRIPTION

The present invention relates to methods of increasing immunogenic cell viability by administering an immunogenic cell and a complement inhibitor to a subject. Another aspect of the invention provides immunogenic cells such as stem cells protected with a coating of complement inhibitor.

### Definitions

For clarification in understanding and ease in reference a list of terms used throughout the brief description section and the remainder of the application has been compiled here. Some of the terms are well known throughout the field and are defined here for clarity, while some of the terms are unique to this application and therefore have to be defined for proper understanding of the application.

“A” or “an” means herein one or more than one; at least one. Where the plural form is used herein, it generally includes the singular.

“Allogenic” as used herein refers to cells from a donor that is different from the recipient (though typically of the same species). Where the donor and the recipient are the same, the cells are “autologous”.

The term “immunogenic” when referring, e.g., to a cell, means that the cell is capable of eliciting a specific immune response, e.g., an innate immune response by complement, against a cell including one or more foreign antigens on its surface. An “immunogenic epitope” is a portion of an antigen to which a specific immune response (e.g., a B cell response and/or a T cell response) is directed, for example, via specific binding of a T cell receptor and/or antibody.

The term “pharmaceutically acceptable,” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “subject”, as used therein, can be a vertebrate or a mammal. Examples of subjects include livestock, test animals, and pets, such as ovine, bovine, porcine, canine, feline and murine mammals, as well as reptiles, birds and fish. The terms, “patient” and “subject” are used interchangeably herein. Preferably, the subject is a mammal. The mammal can be a human, non-human primate, mouse, rat, dog, cat, horse, or cow, but are not limited to these examples.

“Therapeutically effective amount” refers to the amount of an agent determined to produce any therapeutic response in a mammal. For example, an effective combination of immunogenic animal cells and complement inhibitors may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are readily ascertained by one of ordinary skill in the art. Thus, to “treat” means to deliver such an amount.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy. In various embodiments, the symptoms of a disease or disorder are alleviated by at least 5%, at least 10%, at least 20%, at least 30%, at least 40%, or at least 50%.

As used herein, the term “administer” refers to the placement of a composition into a subject by a method or route which results in at least partial localization of the composition at a desired site such that desired effect is produced. An immunogenic cell or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

### Protected Animal Cells

In one aspect, the present invention provides a protected cell. A protected cell, as described herein, is an immunogenic animal cell coated with a complement inhibitor. The cell is protected in vivo because coating the immunogenic cell with the complement inhibitor decreases the likelihood that the immunogenic cell will be destroyed by complement activation. The protected cell has increased viability, and will therefore persist longer in vivo to carry out whatever role is intended for the cell.

One aspect of the invention is directed to an immunogenic animal cell coated with a complement inhibitor. A “complement inhibitor” is a molecule that prevents or reduces activation and/or propagation of the complement cascade. The complement cascade includes a number of targets for pathway inhibition, any of which can be the target of complement inhibitors. Examples of complement inhibitor targets include C1q, C1r, C4, C2, C4b2a, C3, C3a, C3aR, CdbBb, C5, C5a, C5b, C6, C7, C8, and C9. In some embodiments, the complement inhibitor is a molecule that that affects the formation of C3a or signaling through the C3a receptor, or C5a or signaling through the C5a receptor. A complement inhibitor can operate on one or more of the complement pathways, i.e., classical, alternative or lectin pathway. A “C3 inhibitor” is a molecule or substance that prevents or reduces the cleavage of C3 into C3a and C3b. A “C5a inhibitor” is a molecule or substance that prevents or reduces the activity of C5a. A “C5aR inhibitor” is a molecule or substance that prevents or reduces the binding of C5a to the C5a receptor. A “C3aR inhibitor” is a molecule or substance that prevents or reduces binding of C3a to the C3a receptor. A “C4 inhibitor” is a molecule or substance that prevents or reduces the cleavage of C4 into C4b and C4a. A “C1q inhibitor” is a molecule or substance that prevents or reduces C1 q binding to antibody-antigen complexes, virions, infected cells, or other molecules to which C1 q binds to initiate complement activation. See Morgan BP1, Harris C L Nat Rev Drug Discov. 2015 December; 14(12):857-77, the disclosure of which is incorporated herein by reference, which describes a number of complement inhibitors and various points in the complement pathway which are targeted by complement inhibitors.

Complement inhibitors include antibodies (e.g., monoclonal antibodies), small molecules, proteins and peptides, and nucleic acid-based therapy. Examples of complement inhibitors include Compstatin and compstatin analogs (see U.S. Patent Publication 2016/0096870), Vaccinia Virus Complement Control Protein (VCP), C5aR inhibitors such as acetyl-Phe-[Orn-Pro-D-cyclohexylalanine-Trp-Arg] (AcF[OPdChaWR]; PMX-53; Peptech) and analogs of PMX-53 (e.g., PMX-201 and PMX-205; see for instance Proctor et al., 2006, Adv Exp Med Biol. 586:329-45 and U.S. Pat. Pub. No. 20060217530), Neutrazumab, Eculizumab (Alexion Pharmaceuticals, Cheshire, Conn.), Pexelizumab, A R C 1905 (Archemix), Cinryze, Ruconest, Berinert, Heparin, Factor H, NOX-D19, NOX-D20, NOX-21, CCX-168, CF2593A, IFX-1, LFG316, ALXN1210, ALXN5500, SOBI002, Coversin, RA101348, ALN-CC %, C6 antisense or antibody (Regenesance), LampAMY-201, TT30 (ALXN1102), TP10 (CDX-1135), Mirococept, alizumab, NM9401, bikaciomab, OMS721, TNT003, and TNT009.

The immunogenic animal cell is coated with a complement inhibitor. The term “coated,” as used herein, refers to a plurality of complement inhibitor molecules being associated with the surface of the immunogenic animal cell. Complement inhibitor associated with the immunogenic animal cell forms a structure that is sufficiently stable so that the two remain associated under the conditions in which the coating is formed and, preferably, under the conditions in which the coated immunogenic animal cell is used, e.g., physiological conditions. Association includes both non-covalent and covalently-liked association with the surface of the immunogenic animal cell. Coating does not imply that the entire surface of the immunogenic animal cell is covered with complement inhibitor, but rather that at least a portion of the surface of the immunogenic animal cell is covered with complement inhibitor. For example, in some embodiments, 5% of the surface is coated, while in other embodiments 5% to 15%, 15% to 25%, 25% to 35%, 35% to 45%, 45% to 55%, 55% to 65%, 65% to 75%, 75% to 85%, 85% to 95%, more than 95%, or 100% of the surface is coated.

In some embodiments, the complement inhibitor is covalently linked to the immunogenic cell. The term “linked” means that the complement inhibitor is connected with one another through a covalent linkage. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

In some embodiments, the complement inhibitor is directly attached to a functional group of the immunogenic animal cell that is capable of reacting with the complement inhibitor. For example, proteins found on the surface of the immunogenic animal cell include lysines that have a free amino group that can be capable of reacting with a carbonyl-containing group, such as an anhydride or an acid halide, or with an alkyl group containing a good leaving group (e.g., a halide). Proteins also contain glutamic and aspartic acids. The carboxylate groups of these amino acids also present attractive targets for functionalization using carbodiimide activated linker molecules; cysteines can also be present which facilitate chemical coupling via thiol-selective chemistry (e.g., maleimide-activated compounds). In addition, genetic modification can be applied to introduce any desired functional residue, including non-natural amino acids, e.g. alkyne- or azide-functional groups. See Pokorski, J. K. and N. F. Steinmetz Mol Pharm 8(1): 29-43 (2011).

Alternatively, a suitable chemical linker group can be used. A linker group can serve to increase the chemical reactivity of a substituent on either the agent or the virus particle, and thus increase the coupling efficiency. Suitable linkage chemistries include maleimidyl linkers and alkyl halide linkers and succinimidyl (e.g., N-hydroxysuccinimidyl (NHS)) linkers (which react with a primary amine on the filamentous or rod-shaped plant virus particle). Several primary amine and sulfhydryl groups are present on viral coat proteins, and additional groups can be designed into recombinant viral coat proteins. It will be evident to those skilled in the art that a variety of bifunctional or polyfunctional reagents, both homo- and hetero-functional (such as those described in the catalog of the Pierce Chemical Co., Rockford, Ill.), can be employed as a linker group. Coupling can be affected, for example, through amino groups, carboxyl groups, sulfhydryl groups or oxidized carbohydrate residues. For example, in some embodiments, EDC/NHS is used to covalently coat the immunogenic animal cell with the complement inhibitor.

In one aspect, the present invention is directed to immunogenic animal cells coated with a complement inhibitor. Animal cells can be administered to a subject for a wide variety of different reasons, and a wide variety of different types of animal cells can be used. The animal cells can be vertebrate or invertebrate animal cells, mammalian cells, or in some embodiments human cells. The animal cells can be autologous cells that are immunogenic as a result of differences in the cells that arise from cell culturing, or can be immunogenic as a result of being xenogenic or allogenic cells. In some embodiments, the immunogenic cells are autologous or allogenic cells (e.g., stem cells). Examples of cells that can be administered to a subject include stem cells, epithelial cells, pituitary cells, thyroid cells, gut cells, respiratory tract cells, pancreatic cells, nerve cells, immune cells (e.g., β-cells, T-cells, neutrophils, monocytes), kidney cells, adipocytes, fibroblasts, chondrocytes, heart cells, muscle cells, blood cells, and germ cells.

In some embodiments, the immunogenic animal cells are stem cells. Stem cells are undifferentiated biological cells that can differentiate into specialized cells and can divide (through mitosis) to produce more stem cells. Stem cells can undergo self-renewal (i.e., progeny with the same differentiation potential) and also produce progeny cells that are more restricted in differentiation potential. In mammals, stem cells are divided into embryonic stem cells and adult stem cells. Embryonic stem cells refer to stem cells derived from the inner cell mass of blastula stage embryos, while adult stem cells reside in specialized regions, niches, within the different organs or tissues of the adult organism. While the defining difference between the two categories is based on the site of origin of the stem cell, these differences lead to important functional differences. Most important of the fundamental differences deals with the competence of the stem cells to generate different kinds of tissues or cell types. Adult stem cells reside within specialized niches of the adult organs and are multipotent meaning that they are capable of differentiating into a limited number of cell types, usually representative of the different cell types present within the organ of origin. Embryonic stem cells are pluripotent cells capable of differentiating into all the cell types of the adult organisms. This capability is reflective of their natural role in development. The migration of the inner cell mass through the primitive streak during gastrulation establishes the formation of the three embryonic germ layers: ectoderm, mesoderm and endoderm. In turn these three germs layers will develop into all of the tissues and organs present within the body. Ectoderm is destined to develop into the nervous system and the epidermis. Mesoderm forms the hematopoietic system, connective tissue, heart, kidneys, muscular and skeletal systems. While the endoderm will give rise to the gut tube and all of the organs that are derived from it including; the trachea, lungs, thyroid, stomach, liver, pancreas, intestines and colon.

Stem cells can also be characterized in terms of differentiation potential and/or tissue source as pluripotent, multipotent, induced pluripotent, and mesenchymal stem cells. The stem cells used in the invention may be autologous with respect to the subject to be treated. In some embodiments, the stem cells may be allogeneic or xenogeneic with respect to the subject to be treated. Allogenic stem cells derived from a donor may theoretically be used for the treatment of any patient, irrespective of MHC incompatibility.

“Embryonic stem cells (ESCs)” refers to cells that have unlimited self-renewal and multipotent differentiation potential and are derived from the inner cell mass of the blastocyst or can be derived from the primordial germ cells of a post-implantation embryo (embryonal germ cells or EG cells). ES and EG cells have been derived, first from mouse, and later, from many different animals, and more recently, also from non-human primates and humans. When introduced into mouse blastocysts or blastocysts of other animals, ESCs can contribute to all tissues of the animal. ES and EG cells can be identified by positive staining with antibodies against SSEA1 (mouse) and SSEA4 (human). See, for example, U.S. Pat. Nos. 5,453,357; 5,656,479; 5,670,372; 5,843,780; 5,874,301; 5,914,268; 6,110,739 6,190,910; 6,200,806; 6,432,711; 6,436,701, 6,500,668; 6,703,279; 6,875,607; 7,029,913; 7,112,437; 7,145,057; 7,153,684; and 7,294,508, each of which is incorporated by reference for teaching embryonic stem cells and methods of making and expanding them. Accordingly, ESCs and methods for isolating and expanding them are well-known in the art.

“Multipotent” or “multipotent cell” refers to a cell type that can give rise to a limited number of other particular cell types. Multipotent cells are committed to one or more embryonic cell fates, and thus, in contrast to pluripotent cells, cannot give rise to each of the three embryonic cell lineages as well as extraembryonic cells.

“Pluripotent” is meant that the cell can give rise to each of the three embryonic cell lineages as well as extraembryonic cells. Sources of pluripotent cells include cultures derived from the inner cell mass of blastula staged embryos as well as induced pluripotent stem cells (IPS). Pluripotency as used herein is limited to the inner cell mass state, and is not used at any point to describe lineage restricted progenitors that form during development. The pluripotent cell state is a natural one, but can be propagated in-vitro under specific conditions known to those skilled in the art. In such culture conditions, perpetual maintenance of the stem cell, or pluripotent state, occurs. The defining qualities of the pluripotent cells are not limited to their functional characteristic, but can also be described through gene expression patterns and consequently the identification of pluripotent cells can be accomplished through staining patterns for markers they exhibit. The presence of the protein transcription factors OCT3/4, SOX2 and NANOG represent a group of core transcription factors involved in maintaining the pluripotent state and the down regulation of these factors is a prerequisite for forward differentiation. These three transcription factors have all been shown to interact with one another and there is a great deal of overlap in the genes that are under their transcriptional control. In addition to the transcription factors responsible for pluripotent maintenance are often identified through the use of several surface markers present on them, the most common ones being Stage Specific Embryonic Antigens 3 and 4 (SSEA3 and SSEA4), Tra-1-61 and Tra-1-81. Pluripotent cells, such as embryonic stem cells, can also exhibit alkaline phosphatase activity which is often used in the identification of pluripotent cells.

“Induced pluripotent stem cells (IPSC or IPS cells)” are somatic cells that have been reprogrammed, for example, by introducing exogenous genes that confer on the somatic cell a less differentiated phenotype. These cells can then be induced to differentiate into less differentiated progeny. IPS cells have been derived using modifications of an approach originally discovered in 2006 (Yamanaka, S. et al., Cell Stem Cell, 1:39-49 (2007)). For example, in one instance, to create IPS cells, scientists started with skin cells that were then modified by a standard laboratory technique using retroviruses to insert genes into the cellular DNA. In one instance, the inserted genes were Oct4, Sox2, Lif4, and c-myc, known to act together as natural regulators to keep cells in an embryonic stem cell-like state. These cells have been described in the literature. See, for example, Wernig et al., PNAS, 105:5856-5861 (2008); Jaenisch et al., Cell, 132:567-582 (2008); Hanna et al., Cell, 133:250-264 (2008); and Brambrink et al., Cell Stem Cell, 2:151-159 (2008). These references are incorporated by reference for teaching IPSCs and methods for producing them. It is also possible that such cells can be created by specific culture conditions (exposure to specific agents).

During development, mesenchyme forms the connective tissue between and within the developing tissues and organs. Further, mesenchyme has been implicated in secreting signals needed for the growth and differentiation of developing organs. No better example of the importance of mesenchymal cells in development can be offered than the stem cell niche. The niche is the environment which helps sustain stem cells in an undifferentiated state; it is the surrounding microenvironment that stem cells reside in which is composed of extracellular components as well as adjacent cellular components. While the existence of organ-specific stem cells is debated depending on the organ in question, there are some well characterized stem-cell niches within mammalian systems including the hematopoietic stem cell niche and the intestinal crypt stem cell niche. Both cases have a common theme between them being the association of stromal or mesenchymal cells respectively within these niches. These associated cells have been implicated in the regulation of cell activation, proliferation and differentiation of the actual stem cells making them an integral functional component of the niches.

Mesenchymal cells are characterized by their ability to produce and secrete structural molecules commonly found within the extra cellular matrix (ECM), though it is well established that mesenchymal cells also produce an array of different signaling molecules which can function in both the differentiation of cells (as in the case of the developing organ) as well as the maintenance of stemness, as seen in the mesenchymal components of the intestinal stem-cell niche. The expression patterns of mesenchymal cells have been shown to be vastly different depending on the site of origin, though some general markers expressed in multiple mesenchymal lineages are vimentin, fibronectin and various forms of collagen. In contrast to the cells of the epithelial components of a developing organ mesenchyme is marked by a migrating capability.

“Mesenchymal stem cells” or “MSCs” are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999).

### Methods of Increasing Cell Viability

Another aspect of the invention provides a method of increasing immunogenic cell viability by administering an immunogenic animal cell and a complement inhibitor to a subject. The complement inhibitor improves cell viability by protecting the cell from complement-mediated cell destruction. A variety of degrees of improvement in cell viability can be obtained. Wherein the immunogenic cells are viable for at least twice as long as corresponding immunogenic cells administered to a subject without a complement inhibitor.

In some embodiments, the complement inhibitor is merely co-administered to the subject along with the immunogenic animal cells, while in other embodiments the immunogenic animal cell is coated with the complement inhibitor (e.g., the complement inhibitor is covalently linked to the immunogenic animal cell).

The immunogenic cell and the complement inhibitor used in the method can be any combination of the immunogenic animal cells and complement inhibitors described herein. For example, in some embodiments, the immunogenic animal cell is an autologous or allogenic stem cell, while in other embodiments the immunogenic animal cell is a mesenchymal stem cell. Particular examples of complement inhibitors include heparin and factor H. In some embodiments, the immunogenic cell is an autologous or allogenic mesenchymal stem cell, and the complement inhibitor is heparin or factor H.

Immunogenic animal cells can be administered to a subject for a variety of purposes. In some embodiments, the subject (e.g., a human subject) is in need of cell transplantation. In other embodiments the subject (e.g., a human subject) is in need of autologous or allogenic stem cell transplantation. However, cells, and in particular stem cells, can be administered to a subject to treat a variety of conditions. For example, cells can be administered to a subject needing treatment for diabetes, rheumatoid arthritis, Parkinson's disease, Alzheimer's disease, osteoarthritis, stroke and traumatic brain injury, spinal cord injury, heart infarction, cancer, tooth replacement, defective hearing, eye repair, amyotrophic laterial sclerosis, Crohn's disease, infertility, and wound healing.

In some embodiments, the complement inhibitor is not chemically associated with the immunogenic animal cell, but instead is simply co-administered with the immunogenic animal cell. Co-administration includes concurrent and colocational administration.

The term “concurrent” or “concurrently” means at or about the same time. Concurrent administration of an immunogenic animal cell together with a complement inhibitor means administration of the two at or about the same time. In some embodiments, concurrent administration with the combination is simultaneous. However, in other cases, administration of the immunogenic animal cell and the complement inhibitor can be separated in time. Typically, concurrent administration occurs no longer than about 2 days apart, preferably no later than about 1 day, such as within about 8 hours or less. Commonly, concurrent administration of two or more immunogenic components occurs within about 2 hours or less, such that the immunogenic animal cell and the complement inhibitor are administered within a period of 2 hours, a period of 1 hour, or within about 30 minutes, or about 10 minutes. In some instances, concurrent administration is performed at the same time, e.g., in one or more injections.

The term “colocationally” means that the immunogenic animal cell and the complement inhibitor are administered to the same (or about the same) location on the body of the recipient subject. The same location will be understood herein to mean to the same or approximately the same site or orifice. For example, in the case of mucosal administration, an immunogenic combination can be administered to the same orifice (e.g., the mouth or nose). In the case of parenteral administration, colocationally means in proximity at the same (or approximately the same) site on the body, such as to the same site (e.g., by the same device), or within about 10 cm, or more commonly within about 5 cm, such as within about 2 cm, or within 1 cm. In some instances, the two or more components are combined (co-formulated) in a single composition for administration to the same site.

### Dosage and Administration

In some embodiments, the immunogenic animal cells and complement inhibitor can be administered and dosed in accordance with good medical practice, taking into account the site and method of administration, scheduling of administration, patient age, sex, body weight, the nature and severity of the disorder to be treated or prevented, and the form of the complement inhibitor and other factors known to medical practitioners. The cells and/or complement inhibitor may be administered in a single dose or in divided doses. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art.

Typically said dose is about 10×10⁶ cells/kg of subject weight or lower, is about 9×10⁶ cells/kg or lower, is about 8×10⁶ cells/kg or lower, is about 7×10⁶ cells/kg or lower, is about 6×10⁶ cells/kg or lower, is about 5×10⁶ cells/kg or lower. In an alternative embodiment said dose may be between about 0.25×10⁶ cells/kg to about 5×10⁶ cells/kg; or more preferably about 1×10⁶ cells/kg to about 5×10⁶ cells/kg. Accordingly in further alternative embodiments the dose may be about 0.25×10⁶ cells/kg, 0.5×10⁶ cells/kg, 0.6×10⁶ cells/kg, 0.7×10⁶ cells/kg; 0.8×10⁶ cells/kg; 0.9×10⁶ cells/kg; 1.1×10⁶ cells/kg; 1.2×10⁶ cells/kg; 1.3×10⁶ cells/kg; 1.4×10⁶ cells/kg; 1.5×10⁶ cells/kg; 1.6×10⁶ cells/kg; 1.7×10⁶ cells/kg; 1.8×10⁶ cells/kg; 1.9×10⁶ cells/kg or 2×10⁶ cells/kg. The dose may, in other embodiments, be between 0.1 and 1 million cells/kg; or between 1 and 2 million cells/kg; or between 2 and 3 million cells/kg; or between 3 and 4 million cells/kg; or between 4 and 5 million cells/kg; or between 5 and 6 million cells/kg; or between 6 and 7 million cells/kg; or between 7 and 8 million cells/kg; or between 8 and 9 million cells/kg; or between 9 and 10 million cells/kg.

In an alternative embodiment the cells may be administered to the patient as a fixed dose, independent of patient weight. Typically said dose is between about 10 million cells and 500 million cells, e.g. said dose is about 10×10⁶ cells, 50×10⁶ cells, 10×10⁷ cells, 50×10⁷ cells.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

In some embodiments, the immunogenic animal cells and complement inhibitor may be administered in any physiologically acceptable excipient, where the stem cells may find an appropriate site for regeneration and differentiation. In some embodiments, the cells can be frozen at liquid nitrogen temperatures and stored for long periods of time, being capable of use on thawing. If frozen, the cells will usually be stored in a 10% DMSO, 50% FCS, 40% RPMI 1640 medium. Once thawed, the immunogenic animal cells may be expanded by use of growth factors and/or feeder cells according to methods known to those skilled in the art.

In other embodiments, a population of immunogenic animal cells (e.g., stem cells) can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of immunogenic animal cells will be adapted in accordance with the route and device used for administration. In some embodiments, a composition comprising a population of immunogenic animal cells can also comprise or be accompanied with one or more other ingredients that facilitate the engraftment or functional mobilization of the immunogenic animal cells. Suitable ingredients include matrix proteins that support or promote adhesion of the immunogenic animal cells, or complementary cell types, especially endothelial cells. In another embodiment, the composition may comprise resorbable or biodegradable matrix scaffolds.

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1: Local Inhibition of Complement Improves Mesenchymal Stem Cell Viability and Function after Administration

In this report, the inventors identified an antigen on in vitro propagated MSCs that is recognized by the naturally occurring antibodies and developed a simple and economical method to locally inhibit complement on MSCs and protect the cells after administration.

### Results

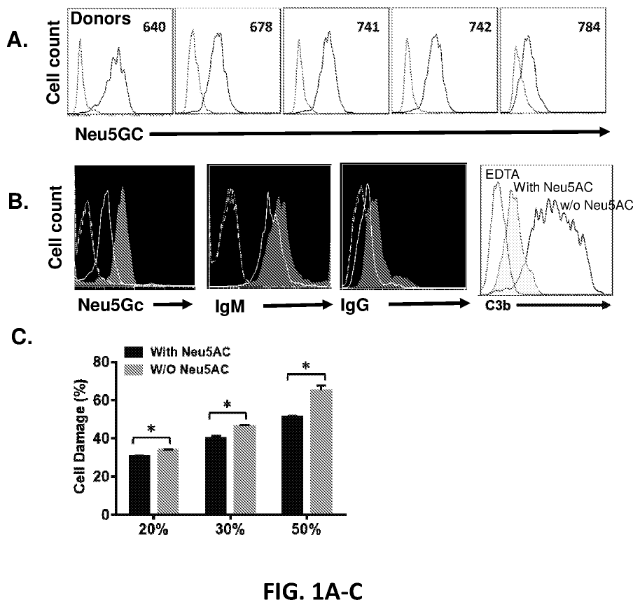

Neu5GC is present on in vitro propagated MSCs. Although the inventors and others have demonstrated that in vitro propagated MSCs spontaneously activate complement upon contact with blood (Moll et al., PLoS One 2011, 6(7): e21703), a process in which pre-existing antibodies are integrally involved, the underlying mechanism remains unclear. Neu5GC is a sialic acid molecule found on cells in most mammals except humans, and almost all humans develop anti-Neu5GC antibodies due to exposure to Neu5GC from different sources, including animal meat consumption. Varki A., American journal of physical anthropology 2001, Suppl 33: 54-69. It was therefore possible that these antibodies might be responsible for the observed complement activation by in vitro propagated human MSCs if the cells took up Neu5GC during culture. To examine whether Neu5GC was present on in vitro propagated human MSCs, MSCs from five donors were incubated with fish gelatin (blocking), stained with chicken anti-Neu5GC Y antibodies and FITC-labeled anti-IgY antibodies and the staining examined by flow cytometry. As shown in FIG. 1A, Neu5GC was found to be present on all tested in vitro propagated MSCs.

Neu5GC contributes to MSC-initiated complement activation. To test the significance of Neu5GC on the MSC surface in MSC-initiated complement activation, MSCs were cultured in complete medium in the absence or presence of N-acetylneuraminic acid (Neu5AC) for 7 days. Consistent with previous reports (Ghaderi et al., Nat Biotechnol 2010, 28(8): 863-867), the addition of Neu5AC to the culture medium significantly reduced Neu5GC levels on the MSC surface (FIG. 1B; left panel). These cells were then incubated with pooled normal human serum and the presence of human IgGs and IgMs on the MSCs was examined by flow cytometry. As shown in FIG. 1B, the results show that human serum immunoglobulins (both IgG and IgM) were bound to the MSCs and that reducing Neu5GC levels on MSCs led to a decrease in the amount of bound IgM and IgG (middle panels). These MSCs were also tested for levels of complement activation in a cell surface C3b deposition assay and in complement-mediated cell damage assays. It was determined that MSCs with lower Neu5GC levels showed C3b deposition (FIG. 1B, right panel) and less complement-mediated injury than those with higher levels (FIG. 1C), suggesting that Neu5GC on MSCs contributes to MSC-initiated complement activation, and, consequently, complement-mediated MSC damage.

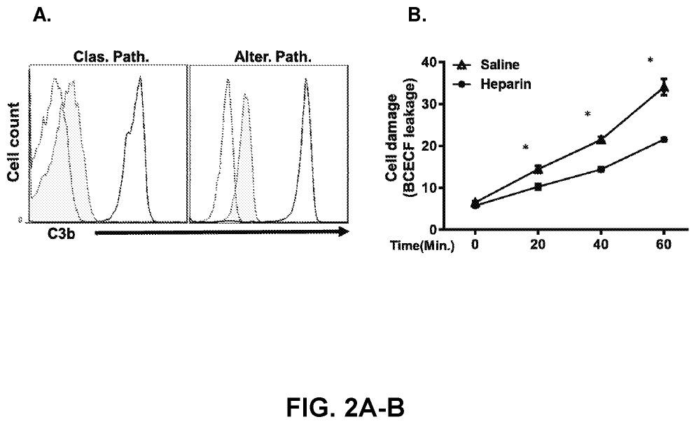

Systemic administration of heparin protects MSCs from serum-mediated attack. Previous studies have shown that heparin is a potent complement inhibitor. Kazatchkine et al., J Exp Med 1979, 150(5): 1202-1215. To test whether it could protect MSCs after their contact with serum, WT C57BL/6 mice were first injected s.c. with either saline or heparin at a dose of 20 U/mouse, then bled 30 min later and complement activity in the plasma was measured using a standard zymosan C3 uptake assay (alternative pathway) and an antibody-sensitized sheep erythrocytes (EshA) C3 uptake assay (classical pathway) in the presence or absence of 1 mM EDTA (EDTA completely inhibits complement activation). As shown in FIG. 2A, these ex vivo assays demonstrated that administration of heparin significantly inhibited complement activity in vivo, as demonstrated by markedly reduced C3 deposition on the surface of both EshA and zymosan. After confirming that heparin inhibited complement activity in vivo, the experiment was repeated, but, this time, BCECF-labeled MSCs were infused into the heparin-treated and control mice via the tail vein 10 min after administration of heparin or saline and in vivo MSC cell damage was examined by measuring the amount of released BCECF in the plasma at different time points after infusion. As shown in FIG. 2B, these results showed that, consistent with the results of the ex vivo complement assay, systemic administration of heparin protected MSCs from serum (complement)-mediated damage in vivo.

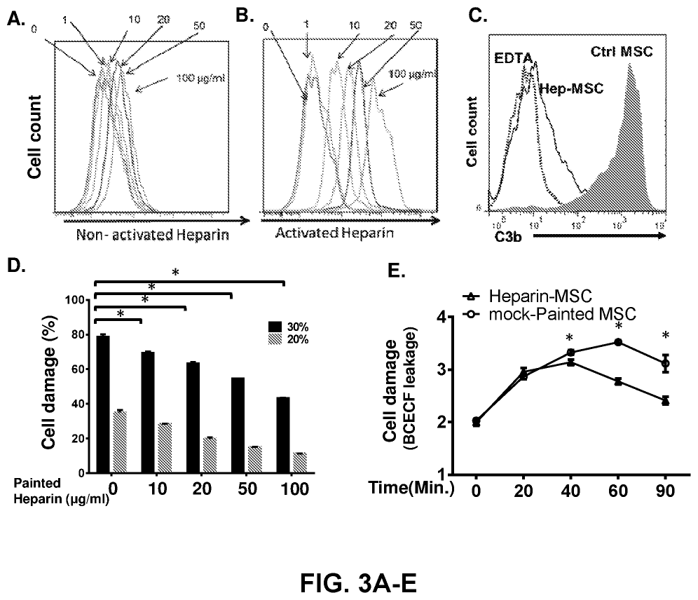

Heparin can be painted onto the MSC surface. The above studies showed that systemic administration of heparin inhibited complement activation and protected MSCs after infusion. However, this approach could raise problems, as heparin not only inhibits complement, but also has strong anti-coagulating activity and systemic administration of large doses of heparin might lead to many side effects, including hemorrhage. In addition, systemic inhibition of complement increases the risk of opportunistic infections. Thus, it would be much better if the heparin could be targeted onto the MSC surface to locally inhibit complement on MSCs, rather than systematically inhibit complement in the blood. To achieve this, heparin was first labeled with FITC, the labeled heparin was then activated using EDC/NHS. To paint the MSCs, MSCs were incubated for 30 min at 37° C. with 1, 10, 20, 50, or 100 μg/ml of activated or non-activated labeled heparin, then, after washing, the cells were examined for cell surface-bound heparin by flow cytometry. As shown in FIG. 3A, at high concentrations (50 and 100 μg/ml), some binding of non-activated heparin to MSCs was observed; however, as shown in FIG. 3B, activation of heparin resulted in a marked increase in binding to the MSC surface and the effect was dose-dependent.

Heparin-painted MSCs are protected from serum-mediated attack. To examine whether painted heparin locally inhibited complement activation on MSCs and protected the cells from complement-mediated attack, one set of MSCs was painted with 100 μg/ml of heparin and a second set was mock-painted, the cells were then incubated for 30 min at 37° C. with 30% normal human serum in GVB++ in the presence or absence of 1 mM EDTA. C3b/iC3b deposition on the MSCs was then assessed by flow cytometry and it was found that, heparin-painted MSCs showed markedly less C3b/iC3b deposition on the cell surface than control MSCs (FIG. 3C). The BCECF release assay was also used to examine the extent of serum-mediated damage to MSCs painted with different concentrations of heparin (0-100 μg/ml) using 20% or 30% normal human serum and it was found that heparin painting resulted in less damage (FIG. 3D). Finally, BCECF-loaded MSCs with or without painting with 100 μg/ml of heparin were adoptively transferred into WT C57BL/6 mice via the tail vein and in vivo MSC injury was evaluated by measuring levels of released BCECF in the plasma at time points from 0 to 90 min, and found that heparin-painted MSCs showed significantly less damage in vivo (FIG. 3E). These data therefore suggest that painting heparin onto the MSC surface might be a valid strategy for locally inhibiting complement activation on MSCs and protecting them from complement-mediated attack.

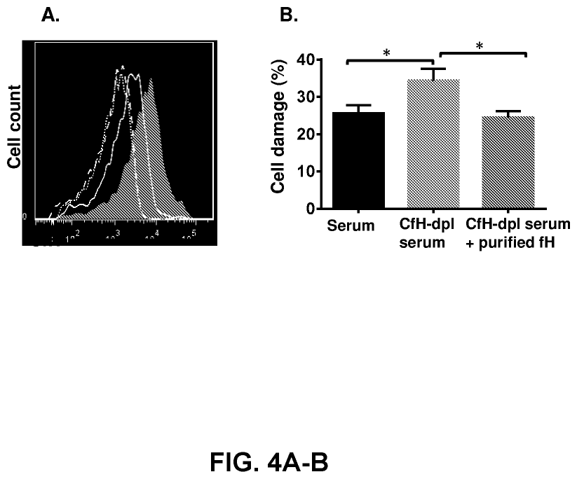

Heparin painted on MSCs recruits factor H from the serum onto the MSC surface. Heparin not only directly inhibits complement, but can also bind factor H21, the most potent native complement inhibitor in serum. To explore the potential mechanisms by which painted heparin inhibited complement activation on MSCs, MSCs were incubated with or without 100 μg of EDC/NHS-activated heparin, the mock-painted and heparin-painted MSCs were then incubated for 30 min with 30% normal human serum or 30% factor H-depleted serum (control) in the presence of 1 mM EDTA (to avoid activation of complement). After washing and staining the cells with anti-factor H antibodies the levels of cell surface-bound factor H on the MSCs was examined by flow cytometry. As shown in FIG. 4A, compared with MSCs incubated with factor H-depleted serum (dotted and dashed lines), both heparin-painted and mock-painted MSCs had factor H bound on their cell surface. However, the heparin-painted MSCs had significantly more factor H bound to their surface after incubation with normal human serum (shaded area) than control MSCs after their incubation with normal human serum (solid line), showing that heparin painted on MSCs recruited factor H onto the MSC surface.

Factor H recruited from serum is involved in protecting MSCs painted with heparin. To test whether the heparin-recruited factor H on MSC cell surface was involved in protecting MSCs from serum-mediated attack, heparin-painted (100 μg/ml) MSCs were incubated for 30 min with (i) 30% normal human serum in PBS, (ii) 30% factor H-depleted human serum in PBS, or (iii) 30% factor H-depleted human serum in PBS supplemented with 250 μg/ml of purified human factor H. To ensure complement activation did not occur during this incubation, 1 mM EDTA was included in the PBS. After washing, the cells were labeled with BCECF, and then were incubated for 30 min with 30% factor H-depleted serum in GVB++ to allow complement activation to occur without recruitment of any additional factor H from the serum, then MSC injury was measured using the BCECF release assay. As shown in FIG. 4B, heparin-painted MSCs that recruited factor H from normal human serum (left column) or from factor H-depleted serum supplemented with factor H (right column) showed less cell damage than those incubated in factor H-depleted serum (center column), demonstrating that heparin-recruited factor H plays an important role in protecting MSCs from complement-mediated damage.

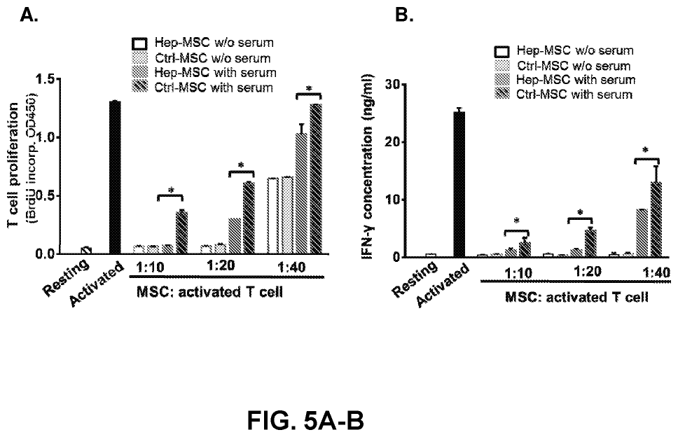

Painting with heparin does not impair the inhibitory activity of MSCs on T cells before contact with serum and preserves it after contact with serum. The above studies demonstrated that heparin-painted MSCs underwent less cellular damage after contact with serum because of the direct complement inhibitory activity of heparin and its ability to recruit complement inhibitor factor H from the serum onto the MSCs. Next, whether, after contact with serum, heparin-painted MSCs showed greater preservation of function than mock-painted MSCs was examined. Heparin-painted or mock-painted MSCs (both treated with mitomycin C to prevent proliferation) were first incubated with 30% normal human serum at 37° C. for 30 min in GVB++. After washes, these serum-incubated MSCs were then incubated with activated mouse T cells at different MSC/activated T cell ratios for 72 h and the ability of the MSCs to inhibit the proliferation of the activated T cells was compared using a BrdU incorporation assay and to inhibit IFNγ production by the T cells using ELISA. As seen in FIG. 5, these experiments demonstrated that, without serum incubation, heparin-painted and mock-painted MSCs showed similar efficacies in inhibiting the proliferation of activated T cells (panel A) and IFNγ production by the activated T cells (panel B), showing that painted heparin did not interfere with the T cell inhibitory activity of MSCs. However, after serum incubation, the heparin-painted MSCs had significantly higher T cell inhibitory activity than the mock-painted MSCs, as demonstrated by the markedly reduced proliferation of activated T cells and IFNγ production.

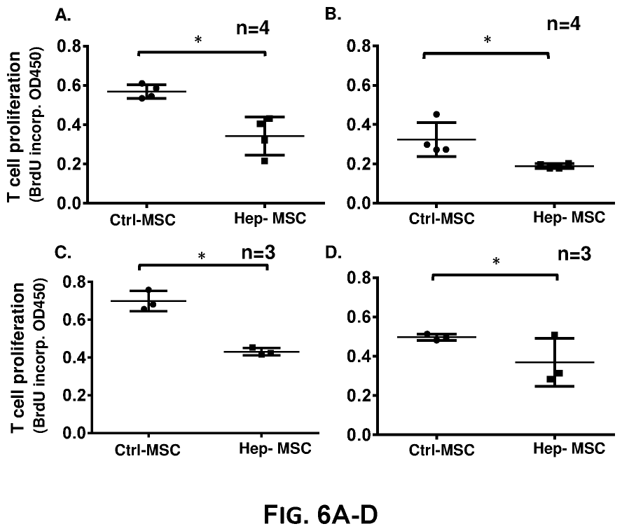

Heparin-painted MSCs are more effective in suppressing T cell function in vivo and survive better than mock-painted MSCs. The above studies suggest that heparin-painted MSCs should be more effective than mock-painted MSCs in inhibiting T cells in vivo. To test this, the efficacy of heparin-painted and mock-painted MSCs in inhibiting the proliferation of activated antigen-specific T cells in vivo was compared. In brief, WT C57BL/6 mice were first injected with OVA-pulsed dendritic cells and OVA323-339-specific T cells (OT-II T cells), then treated with the same number of heparin-painted or mock-painted MSCs, and the efficacy of MSC-mediated T cell inhibition was measured by assessing in vivo OT-II T cell proliferation in the draining lymph nodes (popliteal lymph nodes) using a BrdU incorporation assay. FIG. 6 shows the results from 4 independent experiments (A-D) using the indicated number of mice per group. The results showed that, as suggested by the in vitro studies, heparin-painted MSCs were significantly more effective in suppressing antigen-specific T cell responses in vivo, as indicated by markedly reduced OT-II T cell proliferation in the mice treated with heparin-painted MSCs than those treated with mock-painted MSCs.

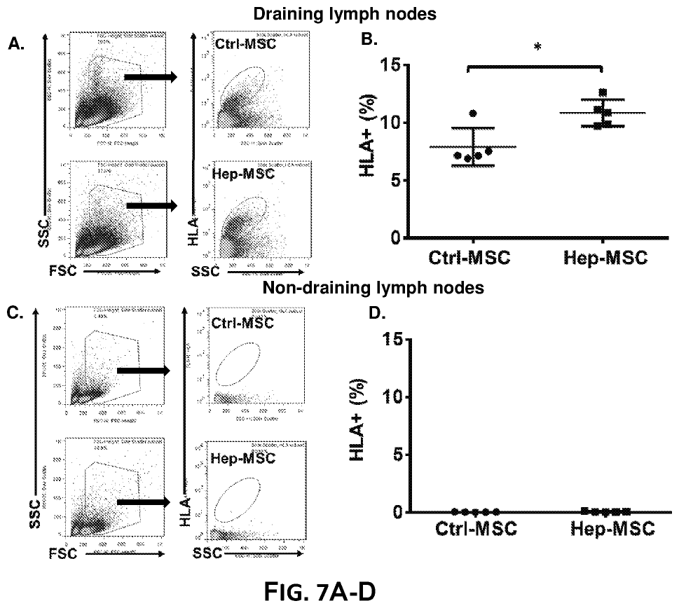

MSC survival in these mice was compared by staining for HLA+ cells in the draining lymph nodes. As shown in FIGS. 7A and B, the percentage of HLA+ cells in the draining lymph nodes was 7.9±1.6% in the mock-painted MSC-injected mice, but 10.8±1.1% in those from heparin-painted MSC-injected mice. As expected, no HLA+MSCs were detected in the distal, non-draining cervical lymph nodes (FIGS. 7C&D). These results show that systemically infused MSCs selectively migrated into the sites of inflammation (draining lymph nodes) and that heparin-painted MSCs survived better in vivo than the control MSCs.

### Discussion

In this study, the inventors demonstrated that Neu5GC incorporated onto bone marrow-derived MSCs during in vitro propagation is recognized by natural IgGs and IgMs in the blood, suggesting that this could be an important mechanism in provoking complement attack on MSCs after administration. The inventors found that heparin, a drug with potent complement inhibitory activity, could be painted onto MSCs and locally inhibit complement activation. Painted heparin did not interfere with the T cell inhibitory activity of MSCs and helped to protect them from complement-mediated attack by directly inhibiting complement on MSCs and by recruiting serum complement inhibitor factor H onto MSCs to indirectly inhibit complement activation. Consequently, both in vitro and in vivo, the heparin-painted MSCs showed better survival and function after contact with complement than mock-painted MSCs.

Due to their strong immunosuppressive activity and their ability to differentiate into different types of cells, MSCs are under intensive development as a new therapy for many inflammatory conditions and regenerative medicine. Currently, more than 500 registered MSC-related clinical trials, but, unfortunately, despite promising pre-clinical study results and excellent safety data from the clinical studies, no MSC-based therapy has yet been approved in the US or Europe. Clearly, current MSC-based therapies need to be improved to be successful.

In both animal and human studies, it is well reported that, after administration, MSCs mysteriously disappear in a short period of time, suggesting that these cells are recognized and attacked by the host. Contrary to the long-held belief that MSCs are immunoprivileged, and can escape host immune surveillance, clinical studies have shown increased levels of the complement activation product C3a in the patient's blood immediately after MSC infusion (Moll et al., Stem Cells 2012, 30(7): 1565-1574; Le Blanc K, Davies L C., Immunol Lett 2015, 168(2): 140-146), indicating complement activation. We have reported that in vitro propagated MSCs activate complement through all three complement activating pathways, and are attacked by MACs, resulting in cell damage and impaired function. However, the mechanism by which complement recognizes MSCs was unclear. In this study, the inventors found that Neu5GC, a sialic acid molecule that exists on the cell surface in almost all mammals, but not humans in vivo, was present on in vitro expanded human MSCs. Humans cannot make Neu5GC due to an inactivating mutation in the gene encoding CMP-N-acetylneuraminic acid hydroxylase, the rate-limiting enzyme in the synthesis of Neu5GC in mammalian cells. Chou et al., Proc Natl Acad Sci USA 1998, 95(20): 11751-11756. Due to exposure to Neu5GC from different sources, including animal meat consumption, most humans develop antibodies against Neu5GC. Thus, the identification of Neu5GC on in vitro expanded MSCs and the observation that MSCs with reduced levels of Neu5GC showed decreased cell damage after contact with serum suggest that Neu5GC incorporated onto MSCs during in vitro propagation, potentially from xenoproteins in the culture medium, is integrally involved in the mechanism by which complement recognizes and attacks MSCs after their administration. However, Neu5GC on in vitro propagated MSCs is not the only factor involved in the triggering of complement activation by MSCs, as markedly reducing Neu5GC levels on MSCs by the addition of Neu5AC to the culture medium only resulted in a moderate reduction in the complement-mediated attack, suggesting that other mechanisms exist and warrant further investigation.

Heparin is a potent complement inhibitor and this effect has been implicated in its clinical benefit in preventing obstetrical complications in pregnant patients with anti-phospholipid syndrome. Girardi et al., Nature medicine 2004, 10(11): 1222-1226. Consistent with previous discoveries, the inventors found that systemic administration of heparin inhibited activation of both the classical and alternative complement pathways. But this approach might result in many side effects, including severe hemorrhage due to the strong anti-coagulation activity of heparin. The inventors' method for painting heparin locally onto MSCs should significantly reduce the amount of heparin required to protect MSCs and result in only local heparin activity on MSCs. In addition, the inventors found that the painted heparin not only directly inhibited complement on MSCs, but also recruited factor H from the serum onto the MSCs to locally inhibit complement. These heparin-recruited factor H seems to play an important but modest role in protecting the MSCs together with the painted heparin, as suggested by the limited difference shown between the cells with or without heparin-recruited factor H. Factor H is present in the serum at a high concentration (˜0.2-0.5 mg/ml) and it is the most potent complement regulator in the serum. Zipfel P F., Semin Thromb Hemost 2001, 27(3): 191-199. These combined direct and indirect complement inhibitory mechanisms might explain the effectiveness of heparin-painting in protecting the MSCs.

In the present study, the inventors used EDC/NHS to activate the heparin to paint the MSCs. EDC/NHS has been used in bioconjugation, crosslinking, labeling, and immobilization applications. Fischer M J., Methods Mol Biol 2010, 627: 55-73. Carboxylate groups on heparin can react with EDC and NHS, resulting in an active NHS ester, which can then react with amine groups on the MSC surface to covalently coat the heparin onto MSCs, a process that the inventors called “painting”. Painting heparin onto MSCs, at least at the doses used in this study, did not interfere with their immunosuppressive activity, as heparin-painted and mock-painted MSCs showed similar potency in inhibiting the proliferation of, and production of IFNγ by, activated T cells. Although the half-life of the painted heparin is relatively short (data not shown), given the hypothesis that MSCs work in a “hit and run” fashion in treating inflammatory diseases, even a modest extension of their “hit” period before they “run” should have a significant impact on their function in vivo. Indeed, the inventors' in vivo studies showed that heparin-painted MSCs survived better in the draining lymph nodes and that they were more potent in suppressing the proliferation of activated antigen-specific T cell responses. In addition, it has been suggested that, after systemic administration, MSCs tend to migrate into sites of inflammation to exercise their functions (Karp et al., Cell Stem Cell 2009, 4(3): 206-216) and this was supported by the inventors' observation that relatively large numbers of systemically administrated (i.v. injection) MSCs were found in the draining lymph nodes (popliteal lymph nodes) after foot pad injection of antigen-pulsed DCs, but not in the distal non-draining lymph nodes (cervical lymph nodes). The inventors also want to point out that, in these in vivo studies, because mice produce Neu5GC, therefore, they do not normally have anti-Neu5GC antibodies, and it is unlikely that complement-mediated MSC damage in these studies was initiated by the anti-Neu5GC IgMs and IgGs shown in the inventors' in vitro studies using human sera. The infused MSCs must be damaged by complement initiated through other similar mechanisms, e.g. natural antibodies against xeno-antigens on the infused MSCs.

In summary, the inventors have found that sialic acid Neu5GC contributes to the mechanism by which MSCs activate complement after administration. The inventors have developed a simple method for painting heparin onto MSCs to locally inhibit complement activation. The painted heparin not only directly inhibits complement on MSCs, but also recruits factor H from the serum to indirectly inhibit complement activation. The inventors also showed, both in vitro and in vivo, that heparin painting does not interfere with the immunosuppressive activity of MSCs and that heparin-painted MSCs have better viability and function after administration. These results suggest that painting heparin onto MSCs could be a simple and effective approach to improve the outcome of current MSC-based therapies.

### Materials and Methods

Animals. OT-II mice (C57BL/6 background) and C57BL/6 mice, purchased from the Jackson Laboratory (Bar Harbor, Me.), were maintained in the animal facility at the Cleveland Clinic. All animal care and experimental procedures were approved by the Institutional Animal Care and Use Committees at the Cleveland Clinic.

MSCs and other reagents. Primary bone marrow-derived human MSCs from healthy donors, provided by the MSC Core Facility at the National Center for Regenerative Medicine (NCRM) of Case Western Reserve University, were cultured in complete medium (α-minimal essential medium containing 16% fetal bovine serum; both from Thermo Fisher Scientific™, Grand Island, N.Y.). Pooled normal human serum and factor H-depleted serum were purchased from CompTech (Tyler, Tex.) and N-acetylneuraminic acid (Neu5AC) was purchased from Nacalai, USA Inc (San Diego, Calif.). The production of the anti-N-glycolylneuraminic acid (Neu5GC) IgY antibodies and control IgY for Neu5GC detection has been described previously. Ghaderi et al., Nat Biotechnol 2010, 28(8): 505 863-867.

Neu5GC detection on MSCs. This was performed as described previously by Ghaderi et al. In brief, after blocking with a 0.5% solution of fish skin gelatin (Sigma-Aldrich, St. Louis, Mo.), MSCs were incubated for 30 min on ice with 5 μg/ml of chicken anti-Neu5GC IgY or control IgY, then were washed, stained with 5 μg/ml of fluorescein isothiocyanate (FITC)-labeled anti-IgY antibody (Jackson ImmunoResearch Laboratories, West Grove, Pa.), and analyzed on a flow cytometer (BD FACSCalibur, San Jose, Calif.).

MSC serum immunoglobulin binding assay. MSCs (0.5×10⁶) cultured in the presence or absence of 3 mM of Neu5AC for a week were incubated for 30 min on ice with 30% pooled normal human serum in PBS. After washing, the cells were stained for cell surface-bound human IgGs and IgMs with 2 μg/ml of anti-human IgG or anti-human IgM antibodies (Jackson ImmunoResearch Laboratories, West Grove, Pa.) and examined by flow cytometry.

Complement alternative pathway and classical pathway activation ex vivo assays. Mice were injected subcutaneously (s.c.) with 20 U of heparin (MP Biomedicals, Solon, Ohio) in saline or saline alone, then blood samples were collected 30 min later and immediately centrifuged for 5 min at 4° C. to collect the plasma. The activity of the complement alternative pathway was assessed using a zymosan C3b deposition assay and complement classical pathway activity was measured using an antibody-sensitized sheep erythrocytes (EshA) C3b deposition assay.

Heparin painting. Heparin was first activated by incubation for 15 min at room temperature with 2 mM 1-ethyl-3-(-3-dimethylaminopropyl) carbodiimide hydrochloride (EDC) and 5 mM N-hydroxysulfosuccinimide (NHS) in MES/NaCl buffer (all from Thermo Fisher Scientific, Grand Island, N.Y.), then 2-mercaptoethanol (final concentration 20 mM) was added to quench the reaction and excess EDC, EDC byproducts, NHS, and 2-mercaptoethanol removed on a PBS-equilibrated desalting column (Thermo Fisher Scientific, Grand Island, N.Y.). The activated heparin was then incubated for 30 min at room temperature with MSCs (100 μg of heparin/10⁶ cells) in 1 ml of HBSS and the cells washed three times with PBS to remove excess free heparin.

To confirm that the activated heparin was painted onto MSCs, in some experiments, the heparin was first labeled with FITC using an FITC labeling kit (Thermo Fisher Scientific, Grand Island, N.Y.) following the manufacturer's protocol, then the FITC-labeled heparin was activated with EDC/NHS and used to paint MSCs as described above and the amount of heparin painted onto MSCs was quantified in a flow cytometer (BD FACSCalibur, San Jose, Calif.). Mock-painted cells went through the same process, but without heparin.

Measurement of C3b deposited on MSCs after contact with serum. Heparin-painted or mock-painted MSCs (0.5×10⁶) were incubated for 30 min at 37° C. with 30% of normal human serum in Gelatin Veronal buffer++(GVB++) without or with 1 mM EDTA (EDTA completely inhibits complement activation). After washes with PBS plus 1% BSA, the cells were incubated with 2 g/mL of FITC-labeled rat anti-human C3 IgG (Cedarlane, Ontario, Canada), washed again and analyzed by a flow cytometer (BD FACSCalibur, San Jose, Calif.). In some experiments, MSCs cultured in media in the absence or presence of 3 mM of Neu5AC for a week were used in the same C3b deposition assays.

Measurement of in vitro damage of MSCs. An established fluorescence dye release assay was used to assess MSC injury after contact with serum. In brief, 2×10⁵ heparin-painted or mock-painted MSCs were first loaded by incubation for 30 min at 37° C. with 5 μM 2′,7′-bis-(2-carboxyethyl)-5-(and-6)-carboxyfluorescein acetoxymethyl ester (BCECF-AM) (Thermo Fisher Scientific™, Grand Island, N.Y.). After 3 washes, the BCECF-loaded MSCs were incubated for 30 min at 37° C. with 20 or 30% normal human serum in 100 μL of GVB++, then the cells were removed by centrifugation and released BCECF in the supernatant measured on a SpectraMax Gemini XS (Molecular Devices, Sunnyvale, Calif.) with excitation and emission wavelengths, respectively, of 485 nm and 538 nm. The MSC damage (percentage of BCECF release) was calculated as ([A−B]/[C−B])×100%, where A represents the mean experimental BCECF release, B the mean spontaneous BCECF release, and C the mean maximum BCECF release induced by incubating the cells with 0.1% Triton-X100.

Measurement of in vivo damage of MSCs. In vivo MSC damage after infusion was also examined using a BCECF release assay. In brief, each mouse was injected subcutaneously with 500 U of heparin in saline or saline alone, then 1×10⁶ MSCs labeled with BCECF were injected by the tail vein, then blood samples were collected after 0, 20, 40, and 60 min and released BCECF in the plasma measured on a SpectraMax Gemini XS (Molecular Devices, Sunnyvale, Calif.) as above.

To evaluate in vivo damage to MSCs with or without heparin painting, 0.5×10⁶ heparin-painted or mock-painted MSCs were labeled with BCECF, then injected into the tail vein and blood sample collected and the plasma assayed for levels of released BCECF as above.

Heparin-recruited factor H assays. Heparin-painted MSCs (0.5×10⁶) were incubated for 30 min on ice with 30% normal human serum or factor H-depleted serum in PBS containing 1 mM EDTA to prevent complement activation. After washing, the cells were incubated with 5 μg/ml of goat anti-factor H IgG (CompTech, Tyler, Tex.) followed by 2 μg/ml of FITC-labeled donkey anti-goat IgG (Jackson ImmunoResearch Laboratories, West Grove, Pa.), then the cells were washed and MSC-bound factor H assessed by flow cytometry.

To examine the function of heparin-recruited factor H, heparin-painted MSCs were first incubated for 30 min on ice with 30% normal human serum, 30% factor H-depleted serum, or 30% factor H-depleted serum supplemented with 250 μg/ml of purified human factor H (CompTech, Tyler, Tex.) in PBS containing 1 mM EDTA. Different sera were used in this step to provide factor H, and EDTA was used in this step to allow factor H recruitment without the occur of complement activation. They were then washed with GVB++, labeled with BCECF, incubated with 30% factor H-depleted serum in GVB++ for 30 min at 37° C., and BCECF release measured as above. Factor H-depleted serum was used in this step as the source of complement to avoid the interference of factor H presented in the normal serum.

MSC in vitro function assay. After treatment of MSCs for 2 h with 10 μg/ml mitomycin C (Sigma-Aldrich, St. Louis, Mo.) to inhibit their proliferation, MSC immunosuppressive activity was examined in vitro by measuring their ability to inhibit the proliferation of anti-CD3/CD28 mAb-activated mouse T cells using a BrdU incorporation assay with a BrdU ELISA kit (Roche, Chicago, Ind.). In addition, the ability of these MSCs to inhibit inflammatory cytokine production by the activated mouse T cells was evaluated by measuring IFNγ levels in the culture supernatants using an IFNγ ELISA kit (Biolegend, CA), following the manufacturer's protocol.

MSC in vivo function and survival assays. The in vivo immunosuppressive activity of MSCs was assessed by measuring their ability to inhibit the proliferation of ovalbumin (OVA)-specific T cells in the draining lymph nodes. In brief, T cells were enriched from OT-II mouse splenocytes using nylon wool and injected via the tail vein into naïve wild-type (WT) C57BL/6 mice (4×10⁶ cells/mouse). After 24 h, syngeneic bone marrow-derived dendritic cells loaded with OVA were injected s.c. into the hind footpads (0.4×10⁶/mouse) and heparin- or mock-painted MSCs (1×10⁶/mouse) were injected via the tail vein. After a further 24 h, 1 mg of BrdU was injected intraperitoneally into each mouse, then, 24 h later, the draining lymph nodes (popliteal lymph nodes) were harvested and the proliferation of the T cells quantified by measuring BrdU incorporation using a BrdU ELISA kit (Roche, Chicago, Ind.).

To evaluate in vivo survival of MSC, in some of the above experiments, draining lymph nodes (popliteal lymph nodes) were collected 48 h after injection of the mock-painted or heparin-painted MSCs and the survival of the injected MSCs assessed by staining the cells with 3 μg/ml of mAb against human HLA-ABC antigen (Clone W6/32, Biolegend, San Diego, Calif.), then measuring numbers of living human cells in a flow cytometer (BD FACSCalibur, San Jose, Calif.). Cells from distal non-draining lymph nodes (cervical lymph nodes) were included as controls.

### Example 2: Painting Factor H onto Mesenchymal Stem Cells Protects the Cells from Complement- and Neutrophil-Mediated Damage

Previous studies have suggested that MSCs are immunoprivileged and can escape from host immune system surveillance partially due to their potent immunosuppressive activities. Because of this belief and other advantages, such as cost and convenience, many MSCs in clinical development are allogeneic MSCs. The inventors and others previously reported that, although MSCs seem to be able to escape from adaptive immune surveillance, infused MSCs can be recognized by complement from the innate immune system. Li Y, Lin F., Blood. 2012; 120:3436-43; Moll et al., Stem cells. 2012; 30:1565-74. Using serum as a source of complement, the inventors further demonstrated that MACs are assembled on MSCs and directly damage the cells, leading to cellular injury and impaired function.

In practice, when MSCs are administrated, they encounter not only complement, but also other cells in the blood. In this study, the inventors investigated the impact of neutrophils, the most abundant circulating leukocytes in the blood in mammals, on the fate of MSCs after administration and the underlying mechanisms. In addition, the inventors also studied the effects of locally “painting” MSCs with complement factor H (CFH) (Whaley K, Ruddy S., J Exp Med. 1976; 144:1147-63), a native complement inhibitor, on their viability and function after administration. The inventors found that MAC generation and activation of neutrophils by C5a synergistically damage MSCs, leading to reduced MSC viability and impaired function. The inventors also demonstrated that the simple CFH painting approach protects MSCs from both MAC-mediated and neutrophil-mediated attack, leading to significantly improved MSC survival and function both in vitro and in vivo.

### Reagents and Methods

MSCs, mice and normal human serum. Primary human MSCs were isolated from bone marrow from healthy donors at the MSC Core Facility at Case Western Reserve University and were used at passage number 4-7 were used. MSCs were cryopreserved in a liquid nitrogen and before each experiment, MSCs were thawed and cultured for 1 week in complete media under standard culturing conditions. Pooled normal human serum (NHS) were ordered from Innovative Research Inc (Novi, Mich.), and aliquoted NHS were stored in a −80° C. freezer until needed. OT-II mice, C3 knockout mice (both are on C57BL/6 background) and C57BL/6 mice, purchased from the Jackson Laboratory (Bar Harbor, Me.), were maintained in the animal facility at the Cleveland Clinic. All animal care and experimental procedures were approved by the Institutional Animal Care and Use Committees of the Cleveland Clinic.

In vivo neutrophil activation assay. 1×10⁶ MSCs in 300 μl of sterile PBS or PBS alone was injected into WT or C3 KO mice by tail vein injection, then, after 40 min, activation of neutrophils in the blood was assessed by staining peripheral blood cells for reactive oxygen species (oxidative burst) with 10 μM dihydrorhodamine 123 (DHR123; Sigma, St. Louis, Mo.) for 20 min at room temperature. Richardson et al., J Immunol Methods. 1998; 219:187-93. After washing and lysis of red blood cells, neutrophil activation (DHR123+) was measured using a flow cytometer (FACSCalibur BD Bioscience).

In vivo neutrophil depletion. Neutrophils were depleted by intraperitoneal injection of mice with an anti-neutrophil monoclonal antibody (NIMP-R14; kindly provided by Dr. Eric Pearlman, Case Western Reserve University) (0.5 mg/mouse) 24 h before the experiment following a previously published protocol (de Vries et al., J Immunol. 2003; 170:3883-9) and neutrophil depletion was confirmed by staining the peripheral white blood cells with phycoerythrin (PE)-labeled anti-mouse Gr-1 monoclonal antibody (mAb) (2 μg/ml Biolegend, San Diego, Calif.) for 15 min at 4° C., followed by flow cytometry analysis.

Complement-mediated MSC damage assay. MSC damage after incubation with serum was assessed using a 2′,7′-bis-(2-carboxyethyl)-5-(and-6)-carboxyfluorescein (BCECF) leakage assay. In brief, 2×10⁴ MSCs were labeled by incubation for 30 minutes at 37° C. with 5 μM BCECF-AM (ThermoFisher, Waltham, Mass.). After washing, the labeled MSCs were incubated 30 minutes at 37° C. with different concentrations of pooled normal human serum (NHS) in 100 μL of GVB++ buffer (0.1% gelatin, 5 mM Veronal, 145 mM NaCl, 0.15 mM CaCl₂ and 0.5 mM MgCl₂, pH 7.3, Complement technology Inc, Tyler, Tex.), then released BCECF in the supernatants was measured by a fluorescence GEMINI XS Microplate Reader (Molecular Devices, Sunnyvale, Calif.) with excitation and emission wavelengths of 485 nm and 538 nm, respectively. To calculate the percentage of BCECF release (complement-mediated injury), the following equation was used as reported previously (Quigg et al., J Immunol. 1995; 154:3437-43): percentage of BCECF release (cell damage) [(A−B)/(C−B)]×100%, where A represents the mean experimental BCECF release, B represents the mean spontaneous BCECF release, and C represents the mean maximum BCECF release induced by incubating cells with 0.1% Triton X-100.

Neutrophil-mediated MSC damage assay. Human neutrophils, either purified from healthy donor peripheral blood mononuclear cells (PBMCs) by Ficoll centrifugation (provided by the Hematopoietic Stem Cells Core Facility at Case Western Reserve University) or differentiated from the neutrophil progenitor cell line HL-60 (ATCC), were used. Collins et al., Proc Natl Acad Sci USA. 1978; 75:2458-62. In some experiments, neutrophil-mediated MSC damage was assessed using a similar BCECF-based assay to that already described. In brief, 2×10⁴ MSCs were labeled with BCECF-AM as described above, then, after washing, the labeled MSC were incubated for 30 min at 37° C. with different numbers of neutrophils in the presence or absence of 30% NHS in 100 μl of GVB++ buffer with various concentrations of the neutrophil function inhibitor wortmannin that inhibits oxidative burst (Sue-A-Quan et al., Journal of cellular physiology. 1997; 172:94-108) (Cayman Chemical, Ann Arbor, Mich.) and released BCECF in the supernatants was measured as described. The percentage BCECF release (cell injury) was calculated as above. In some experiments, a C5aR antagonist (JPE1375, Calbiochem, Billerica, Mass.)[27] or a C3aR antagonist (SB 290157, Calbiochem, Billerica, Mass.)[28] were added, both at 10 M, to assess the role of C3aR and C5aR on neutrophils, while, in other experiments, NHS was omitted and 50 ng/ml of purified C5a (Complement technology Inc, Tyler, Tex.) added instead.

Painting purified CFH onto MSCs. Primary MSCs were painted with purified human CFH (Complement technology Inc, Tyler, Tex.) after 1-ethyl-3-(-3-dimethylaminopropyl) carbodiimide hydrochloride (EDC)-mediated activation of carboxyl groups. In brief, 2 mM EDC and 5 mM N-hydroxysulfosuccinimide (ThermoFisher, Waltham, Mass.) were added to CFH solution at the indicated concentration and allowed to react for 15 min at room temperature to create activated carboxyl groups, then 20 mM 0-mercaptoethanol was added to quench the excess EDC and the activated CFH was separated using a desalting column (ThermoFisher, Waltham, Mass.) equilibrated with PBS. MSCs (2×10⁴) were then incubated with different concentrations of activated CFH for 30 min at 37° C. in 100 μl PBS, then the cells were washed with PBS and used for experiments.

Detection of painted CFH on MSCs. Painted CFH on MSCs were detected by flow cytometric analysis. In brief, 0.5×10⁵ CFH-painted or mock-painted (buffers only) MSCs were stained for 30 min at 4° C. with 2 μg/ml of goat anti-human CFH mAb (Complement technology Inc, Tyler, Tex.) or the same concentration of control goat IgG. After washing, the cells were incubated for 30 min at 4° C. with 1 μg/ml of Alexa 488-labeled donkey anti-goat IgG antibodies (Jackson ImmunoResearch, West Grove, Pa.), followed by flow cytometric analysis on a flow cytometer (BD FACSCalibur).

Painted CFH stability assay. MSCs were painted with CFH (10 μg/ml) following the protocols described above. Levels of painted CFH on the cell surface (mean fluorescence intensity) were quantitated by flow cytometric analysis every 12 hr for the first 36 hr, then daily for up to 5 days.

MSC-activated complement assays. Complement activation by CFH-painted or mock-painted MSCs was evaluated by culturing the MSCs with NHS, then measuring levels of C3b/iC3b on the MSCs and C3a and C5a in the supernatants. In brief, MSCs (2×10⁴) painted with different concentrations of activated CFH or mock-painted were incubated at 37° C. for 30 min with 30% NHS in GVB++ buffer, then the MSCs were collected and C3b/iC3b deposited on the cell surface quantified by staining the cells with fluorescein isothiocyanate-conjugated anti-human C3 IgG (Cedarlane, Burlington, N.C.), followed by flow cytometric analysis, while the supernatants from some of these experiments were collected and C3a and C5a levels measured using an anaphylatoxin cytometric bead array kit (BD Biosciences, San Jose, Calif.) according to the manufacturer's instructions.

MSC in vitro function assay. MSC function was assessed by measuring their ability to inhibit the proliferation of, and cytokine production by, activated T cells. In brief, MSCs (2×10⁴) were irradiated at 30-Gy to inhibit proliferation, then were painted for 30 min at 37° C. in the presence or absence of activated CFH (10 μg/ml). After washing, these cells were incubated with 30% NHS in GVB++ buffer for 30 min at 37° C., washed, and incubated with different numbers of PBMCs (MSC: PBMC ratio 1:10, 1:20 and 1:40) in the presence of anti-CD3/28 Dynabeads (2 μl, ThermoFisher, Waltham, Mass.) and human IL-2 (30 U/ml; Biolegend, CA). After 48 h, BrdU (10 μM) was added to the co-cultures and culture supernatants were collected 72 h later and IFN-r levels measured by conventional ELISA (Biolegend, CA), while the cells were fixed and proliferation of the activated T cells assessed by measuring BrdU incorporation using a BrdU ELISA kit (Roche, Chicago, Ind.).

In vivo MSC damage and survival assays. To evaluate in vivo damage of MSCs after administration, MSCs (1×10⁶) labeled with BCECF-AM were infused via the tail vein into WT mice in which neutrophils had been depleted using anti-NIMP antibody, WT mice which had been injected with the same amount of control IgG, and untreated C3 KO mice. For MSC injury assessment, at 0, 20, 40 and 60 min, the mice were bled from the tails and BCECF levels released from damaged MSCs quantified as described. Mice were then euthanized at 6 h after cell transfer, and the lungs, liver, and spleen collected and single cell suspensions prepared either by collagenase digestion (lungs and livers) or by mashing (spleens), then the cells were stained using 2 μg/ml of Cy5.5-labeled anti-human CD105 mAb (Biolegend, San Diego, Calif.), and the number of surviving infused MSCs inside each organ analyzed by flow cytometry (BD FACSCalibur).

In vivo MSC function assay. The in vivo immunosuppressive activity of MSCs was first assessed by measuring their ability to inhibit systemic antigen-specific T cell responses in mice adaptively transferred with uveitigenic Th17 cells. In brief, following previously published protocols (Tu et al., Invest Ophthalmol Vis Sci. 2012; 53:959-66), WT B6 mice were immunized subcutaneously (s.c.) with interphotoreceptor retinoid-binding protein peptide 1-20 (IRBP₁₋₂₀) in complete Freund's adjuvant (200 μg/mouse), then 2 weeks later, spleen cells were collected and re-stimulated with 10 μg/ml of IRBP₁₋₂₀ peptide in vitro, together with 10 ng/ml of IL-23 (Biolegend, CA). After 72 h of culture, blasting T cells were enriched by Ficoll centrifugation and adoptively transferred into naïve B6 mice by tail vein injection (5×10⁶/mouse), which were then randomly divided into 2 groups. One group received 0.5×10⁵ CFH-painted MSCs and the other received the same number of mock-painted MSCs by tail vein injection, then, after 7 days, the mice were euthanized and splenocytes collected for IL-17a recall assays to compare the Th17 responses in the two groups.

The in vivo immunosuppressive activity of MSCs was also assessed by measuring their ability to inhibit local T cell responses using a dendritic cell (DC)-mediated T cell activation model. Bhatt et al., J Immunol. 2014; 192:5098-108. In brief, T cells were enriched from OT-II mouse splenocytes using nylon wool and injected via the tail vein into naïve WT C57BL/6 mice (4×10⁶ cells/mouse). After 24 h, syngeneic bone marrow-derived DCs loaded with ovalbumin (OVA) were injected s.c. into the hind footpads (0.4×10⁶/mouse) and CFH- or mock-painted MSCs (1×10⁶/mouse) were injected via the tail vein. After a further 24 h, 1 mg of BrdU was injected intraperitoneally into each mouse, then, another 24 h later, the draining lymph nodes (popliteal lymph nodes) were harvested and T cell proliferation measured by BrdU incorporation using a BrdU ELISA kit (Roche, Chicago, Ind.).

Data Analysis. All data were analyzed using the software Statistical Package for Social Sciences (SPSS, version 23.0; SPSS Inc., Chicago, Ill.) and GraphPad Prism (version 6.0; GraphPad software Inc., La Jolla, Calif.). To determine whether groups were statistically different, results were compared using Student's t-tests, ANOVA, and Tukey post-hoc tests. A p value of <0.05 was considered significant.

### Results

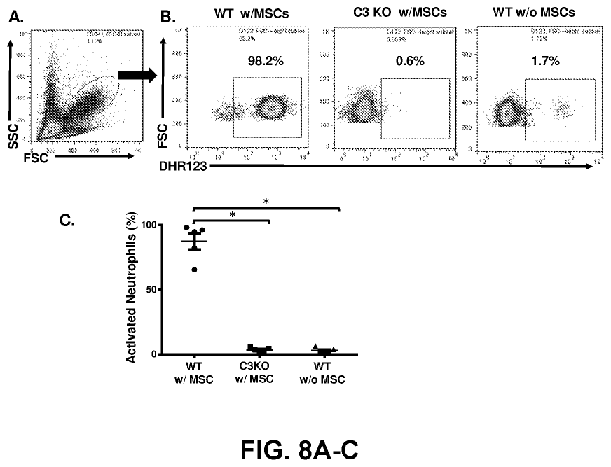

Neutrophils in the blood are activated by complement after MSC administration. To test whether neutrophils were activated after contact of human MSCs with blood following administration and whether complement was involved in this process, because C3 KO mice are most commonly used to demonstrate a role of complement in vivo, MSCs were first infused into WT and C3 KO mice by tail vein injection, then, after 40 min, neutrophil activation was assessed in the peripheral blood by dihydrorhodamine 123 (DHR123) which stains for reactive oxygen species, followed by flow cytometry analysis. Smith J A, Weidemann M J., J Immunol Methods. 1993; 162:261-8. As shown in FIG. 8, the results showed that, while the majority of neutrophils were quiescent in naïve WT mice, after MSC infusion, most neutrophils in the WT mice were activated, as indicated by positive DHR123 staining for reactive oxygen species. In contrast, C3 KO mice showed markedly reduced neutrophil activation after receiving the same numbers of MSCs. These results showed that neutrophils are activated shortly after MSC infusion in vivo and that complement is the primary mechanism by which MSCs activate neutrophils.

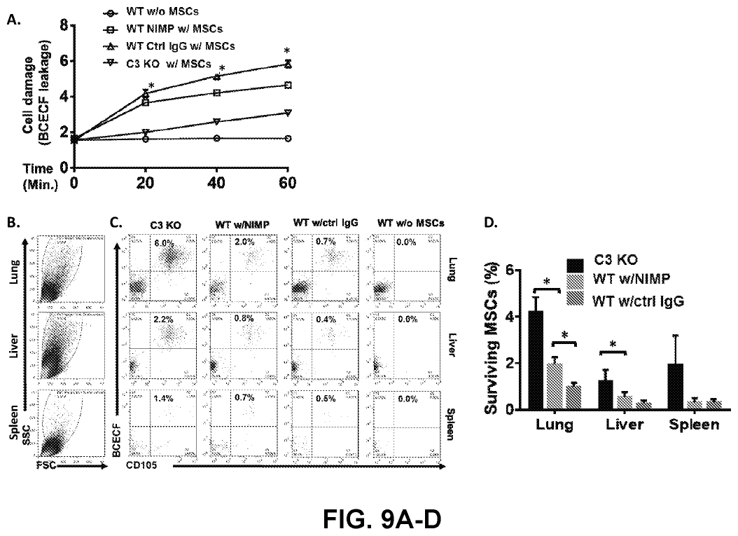

MSCs survive better in C3 KO or neutrophil-depleted WT mice than in control WT mice. The impact of the activated neutrophils on the fate of MSCs after administration was evaluated by measuring MSC injury and surviving MSCs in different organs. MSCs labeled with the fluorescent dye BCECF were infused into C3 KO mice, neutrophil-depleted WT mice (injected with NIMP-R14 anti-neutrophil antibody 24 h before MSC injection), or control IgG-injected WT mice, then, at 0, 20, 40, and 60 min, levels of released BCECF in the blood were measured to monitor in vivo MSCs damage. The mice were then euthanized six hours after MSC infusion and single cell suspensions prepared from the lungs, liver, and spleen and surviving MSCs in these organs measured by flow cytometry by analyzing the percentage of BCECF+CD105+ cells. FIG. 9A shows that the greatest BCECF leakage (more severe damage) from MSCs occurred in the control IgG-injected WT mice, less severe damage was seen with the neutrophil-depleted mice, and the least damage was seen in the C3 KO mice. In accordance with these results, flow cytometric analyses in different tissues showed that more surviving MSCs were found in the C3 KO mice, fewer in the neutrophil-depleted WT mice, and least in the control IgG-treated WT mice (FIG. 9B-D).

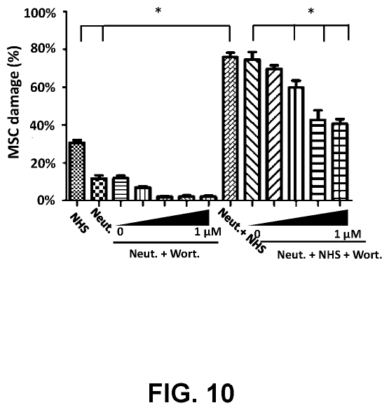

Complement-activated neutrophils and MACs have synergistic effects in damaging MSCs. To elucidate the mechanism underlying the above in vivo results, we carried out in vitro MSC damage assays using neutrophils derived from the progenitor cell line HL-60 in the presence or absence of NHS and in the presence or absence of a known neutrophil function inhibitor, wortmannin, which inhibits oxidative burst. FIG. 10 shows that “quiescent” neutrophils caused some damage to MSCs in the absence of complement (no NHS; left panel) and that this damage was reduced in a dose-dependent manner by wortmannin. When MSCs were incubated with NHS alone, MSCs were damaged by complement membrane attack complexes (left column in left panel), as shown in the inventors' previous report. Li Y, Lin F., Blood. 2012; 120:3436-43. However, when MSCs were incubated with both neutrophils and NHS (right panel), the damage was markedly increased and damage was gradually reduced by increasing concentrations of wortmannin, finally plateauing at a level similar to that seen with NHS alone. These results demonstrate that neutrophils in the blood can act synergistically with complement to damage MSCs after administration.

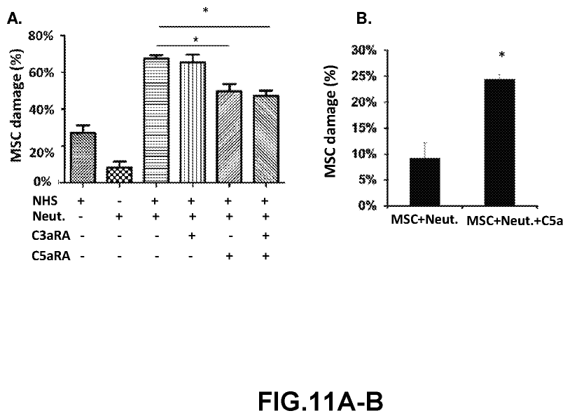

The C5aR, but not the C3aR, on neutrophils is involved in the neutrophil-mediated MSC damage. Neutrophils express both the C3aR and the C5aR. Martin et al., J Exp Med. 1997; 186:199-207. When complement is activated, C3a and C5a, the ligands of these complement receptors, are produced and released into the fluid phase, and can bind to their respective receptor on neutrophils and activate neutrophils to kill. To distinguish the respective roles of C3a and C5a in the newly discovered neutrophil-mediated MSC injury, the HL60-derived neutrophil-mediated and NHS-mediated MSC damage assay was repeated in the presence and absence of a C3aR antagonist and/or a C5aR antagonist. FIG. 11A shows that, although the C3aR antagonist alone had no appreciable effect, the C5aR antagonist caused a significant reduction in neutrophil-mediated MSC damage which was not increased in the presence of the C3aR antagonist, showing that the C5aR, but not the C3aR, on neutrophils contributes to the observed neutrophil-mediated MSC damage.

C5a-activated primary neutrophils damage MSCs. In the above experiments, HL-60-derived neutrophils were used. To test whether primary neutrophils could also induce MSC damage after C5a activation, MSCs were incubated for 4 h with freshly purified primary neutrophils in the absence or presence of purified human C5a, then MSC damage was assessed. FIG. 11B shows that consistent with results obtained using HL-60-derived neutrophils and NHS, primary neutrophils also induced significant MSC damage after stimulation with purified C5a.

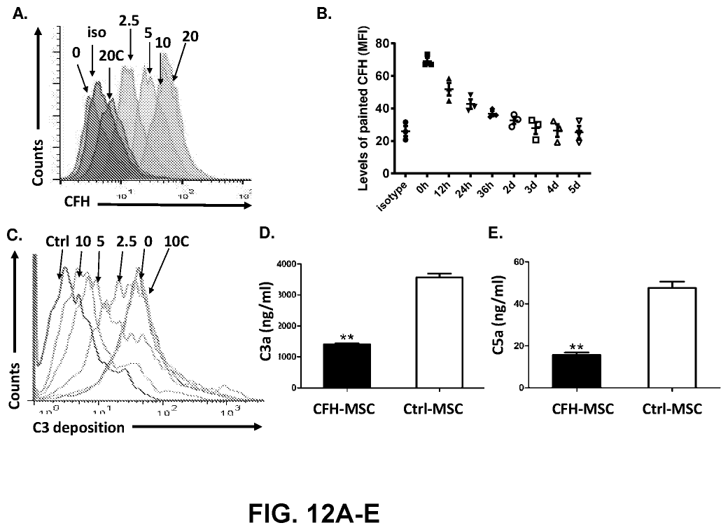

Purified CFH can be painted onto the MSC surface. The above results demonstrate that complement in the blood is activated by the infused MSCs. The activated complement assembles MACs to directly injure MSCs, and, at the same time, the released C5a activates neutrophils in the blood to further damage MSCs. These results suggest that local inhibition of complement activation on MSCs should greatly preserve MSC viability and function by inhibiting not only MAC-mediated, but also neutrophil-mediated, attack. To try to achieve this goal, a protocol for painting CFH onto MSCs was developed in which carboxylic acid groups in the purified CFH were activated with EDC, and were then reacted with amine groups on the MSC surface to covalently cross-link (paint) CFH onto MSCs. FIG. 12A shows flow cytometric results when MSCs were incubated with 0, 2.5, 5, 10, or 20 μg of EDC-activated CFH or 20 μg of non-activated CFH, then with goat anti-CFH antibodies and Alexa 488-labeled donkey anti-goat IgG, demonstrating that activated CFH was bound in a dose-dependent manner. In experiments studying the stability of the painted CFH on MSCs, it was found that the painted CFH can stay on MSCs for 2 days (FIG. 12B).

CFH painted onto MSCs inhibits C3b/iC3b deposition on MSCs and C3a/C5a release after serum contact. To test whether the painted CFH inhibited complement activation on MSCs, MSCs painted with different concentrations of CFH were incubated with or without NHS in GVB++ buffer, then local complement activation was assessed by measuring levels of C3b/iC3b on MSCs using flow cytometry. As shown in FIG. 12C, painting CFH onto MSCs greatly reduced C3b/iC3b deposition on MSCs in a dose-dependent manner. The released C3a/C5a in the supernatants after incubating MSCs painted using 10 μg/ml of activated CFH or mock-painted MSCs with NHS in GVB++ was measured and it was found that levels of released C3a (FIG. 12D) and C5a (FIG. 12E) were significantly reduced in the culture supernatants from CFH-painted MSCs, showing that the painted CFH locally inhibited complement activation on MSCs. In subsequent studies, 10 μg/ml of CFH was used.

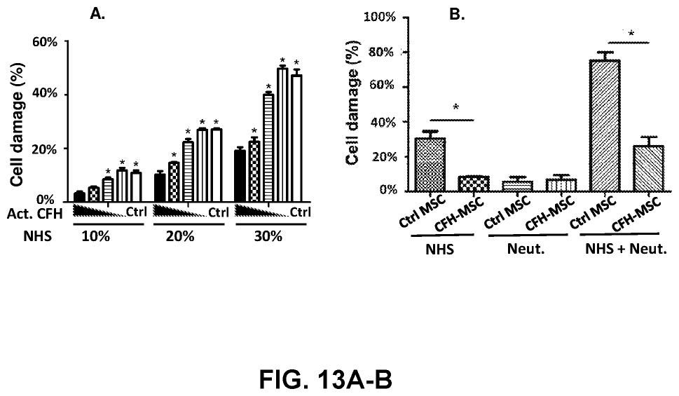

CFH-painted MSCs are better protected from MAC-mediated direct attack than mock-painted MSCs. Whether CFH-painted MSCs were better protected from complement-mediated direct attack than mock-painted MSCs was then tested using the BCECF leakage-based cytotoxicity assay. MSCs were first incubated with 0, 2.5, 5, or 10 μg of activated CFH for painting, MSCs incubated with 10 μg of non-activated CFH were used as controls, then, after washes, the cells were labeled with BCECF, incubated with 10%, 20%, or 30% NHS in GVB++ for 30 min, and MSC damage was measured by measuring levels of released BCECF. As shown in FIG. 13A, painting CFH onto MSCs significantly reduced complement-mediated MSC damage in a dose-dependent manner at all concentrations of NHS (complement) used.

CFH-painted MSCs are also better protected from neutrophil-mediated attack than mock-painted MSCs. Since painting CFH onto MSCs inhibited the production of C5a (FIG. 12D), and since C5a activated neutrophils to further damage the MSCs (FIG. 11), CFH-painted MSCs should also be better protected from neutrophil-mediated damage than mock-painted MSCs. To test this, BCECF-labeled and CFH-painted MSCs or mock-painted MSCs with were incubated NHS alone, neutrophils alone, or both NHS and neutrophils for 4 h, then quantified the damage to MSCs. As shown in FIG. 13B, the results demonstrated that, consistent with previous experiments described above, CFH-painted MSCs showed reduced cellular injury compared to mock-painted MSCs when incubated with NHS alone (left panel). When incubated with neutrophils in the absence of complement (“quiescent” neutrophils), there was no significant difference in terms of MSC injury between the painted and mock-painted MSCs, with little BCECF release in both cases (center panel), but, when incubated with both NHS and neutrophils, the CFH-painted MSCs showed significantly reduced cell injury compared to mock-painted MSCs (right panel), showing that the CFH-painted MSCs are also better protected from attack by complement-activated neutrophils.

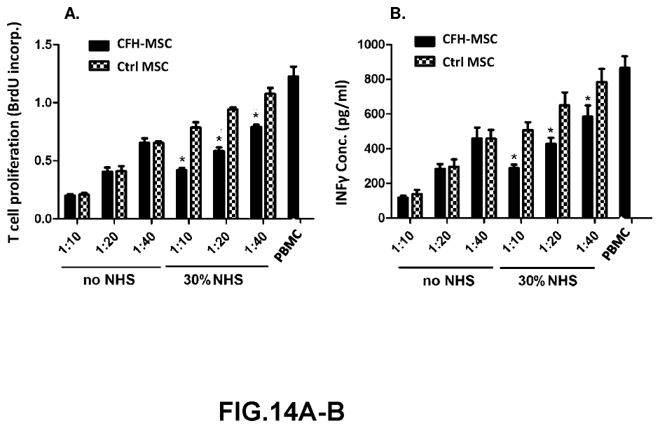

Painting MSCs with CFH does not interfere with their T cell inhibitory activity before contact with serum and preserves MSC function after contact with serum. Whether CFH painting of MSCs affected cell function before and after their contact with complement was then examined. To address this question, CFH-painted MSCs or mock-painted MSCs were incubated for 30 min with 30% NHS in GVB++ or in GVB++ alone, irradiated, and mixed with different numbers of human T cells activated by anti-CD3/CD28 Dyna Beads, then their ability to inhibit the proliferation of, and inflammatory cytokine production by, the activated T cells was compared using BrdU incorporation and IFNγ ELISA. FIG. 14A shows that, at all MSC:T cell ratios tested, CFH-painted MSCs and mock-painted MSCs showed comparable efficacy in inhibiting the proliferation of activated T cells when pre-incubated with buffer alone, and that, after pre-incubation with 30% serum, CFH-painted MSCs were significantly more effective than mock-painted MSCs in inhibiting the proliferation of activated T cells. FIG. 14B shows similar results for IFNγ production by the activated T cells. These data suggest that CFH painting would not interfere with MSC immunosuppressive function before administration and that CFH-painted MSCs would show better retention of function than mock-painted MSCs after administration.

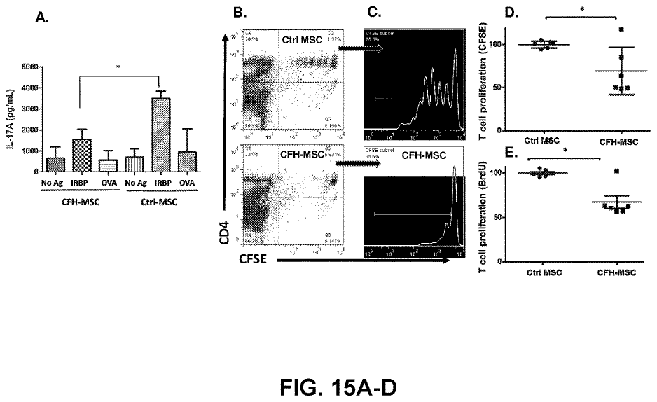

CFH-painted MSCs are more effective at inhibiting in vivo antigen-specific T cell responses than mock-painted MSCs. The above studies strongly suggested that CFH painting of MSCs might be a valid approach for improving the ability of MSCs to inhibit T cell responses in vivo by protecting the cells from complement- and neutrophil-mediated attack. To test this, the ability of CFH-painted MSCs and mock-painted MSCs to inhibit pre-activated antigen-specific T cells systemically transferred into naïve mice was compared. In brief, WT mice were first immunized with IRBP₁₋₂₀ peptide in CFA to induce antigen-specific T cells, then 5×10⁶ in vitro propagated IRBP₁₋₂₀-specific T cells and 0.5×10⁶ CFH- or mock-painted MSCs were adoptively transferred into naïve WT mice by i.v. injection, then, one week later, splenocytes were prepared from the mice and the T cells re-stimulated in the presence of IRBP₁₋₂₀, a non-relevant peptide, or no peptide for 72 h, the IL-17 levels in the culture supernatants were then measured. As shown in FIG. 15A, CFH-painted MSCs were significantly more effective in inhibiting the IRBP₁₋₂₀-specific Th17 response in vivo than mock-painted MSCs.

The in vivo MSC function study was also repeated by comparing the ability of CFH-painted MSCs and mock-painted MSCs to inhibit antigen-specific T cell responses that were locally activated by dendritic cells (DCs) in the draining lymph nodes. In brief, 4×10⁶ OT-II T cells were adoptively transferred into naïve WT C57BL/6 mice, then, one day later, 0.4×10⁶ CFSE-labeled OVA323-339 peptide-loaded DCs were injected into the hind footpads and 1×10⁶ of CFH-painted MSCs or mock-painted MSCs via the tail vein, then, after 48 h, the proliferation of the activated OVA-specific T cells in the popliteal lymph nodes was assessed by flow cytometry (CFSE dilution) and by ELISA (BrdU incorporation). As shown in FIG. 15B-D, at the time point of examination, OT-II T cells in the mice injected with mock-painted MSCs showed extensive proliferation, but this was markedly reduced in mice injected with CFH-painted MSCs. FIG. 15E shows that the above results were supported by the BrdU incorporation results, as significantly lower BrdU incorporation was seen in the draining lymph nodes of the mice injected with CFH-painted MSCs than in those from mice injected with the same number of mock-painted MSCs.

### Discussion

The inventors have previously shown that, after MSCs are infused into the blood, complement is activated, resulting in assembly of MACs to directly damage MSCs. In the present study, the inventors found that, in addition to the direct MAC-mediated attack on MSCs, C5a generated by MSC-mediated activation of complement activated neutrophils in the blood, and that these activated neutrophils have a synergistic effect with MACs in damaging MSCs and impairing their function. The inventors then developed a method for painting CFH onto the surface of MSCs without negatively interfering with MSC function and demonstrated that the painted CFH locally inhibited complement activation on MSCs and reduced C5a release. Consequently, the CFH-painted MSCs showed significantly reduced cell damage, enhanced survival, and improved function both in vitro and in vivo, suggesting that this simple approach could be effective in improving the outcome of current MSC-based therapies for inflammatory diseases.

Largely because of their potent T cell inhibitory activity, MSCs are under intensive clinical development as a new therapy for treating inflammatory diseases. However, despite hundreds of registered clinical trials using MSCs in treating various disease conditions, no product has yet been approved in the US for clinical use. It is also well known that, in both animal and human studies, MSCs rarely engraft and only survive for a very short period of time after administration. As a matter of fact, even the observed short-term survival of MSCs after administration might be an overestimate, because, in most of these studies, MSCs were either labeled with luciferase or fluorescence or isotopes (Weir et al., Heart Lung Circ. 2008; 17:395-403) and residual signals were used to indirectly measure the survival of the cells in vivo; however, damaged MSCs with impaired function or even MSC debris can still retain the label, resulting in false positive signals. In their experiment, the inventors used flow cytometry analysis to gate on “live cells” based on the forward scatter and side scatter patterns, then identified the surviving MSCs by their specific marker (human CD105). This approach should, at least in theory, provide more accurate data reflecting the real status of the injected MSCs than conventional indirect methods measuring residual MSC fluorescence/isotope labels.

The well-documented short life of MSCs after administration and the promising results shown in both human and animal studies led to the “hit-and-run” theory, speculating that, even if MSCs only survive for a short period of time after administration, this limited window is enough to allow them to be beneficial in treated patients/animals through yet-to-be-determined mechanisms. von Bahr et al., Stem cells. 2012; 30:1575-8. This theory, which is gaining more and more ground in the field, suggests that even a small improvement in MSC survival and/or function after administration could have a significant impact on augmenting the efficacy of treatment with MSCs. Indeed, even the painted CFH only stayed on MSC cell surface for up to two days, the inventors found that the improved MSC viability and function after local complement inhibition by painting CFH onto MSCs resulted in significantly increased efficacy of MSCs in inhibiting antigen-specific T cell responses in vivo, suggesting that painting CFH onto the MSCs before administration might be a valid approach for improving MSC viability and function, therefore markedly improving the outcome of current MSC-based therapies.

Although MSCs have been considered to be hypoimmunogenic or immunoprivileged and can escape from host immune surveillance, the inventors and others have reported that MSCs activate complement in the blood, leading to cell damage and impairment of function. In their previous study (Li Y, Lin F., Blood. 2012; 120:3436-43), the inventors focused on the direct effect of complement activation on MSC viability and function, i.e., direct MAC-mediated MSC injury, using serum as the source of complement in the absence of blood cells. However, under physiological conditions, immediately after infusion, MSCs encounter both complement and cellular components in the blood. Neutrophils, the most abundant leukocytes in the blood and an important component of the innate immune cells, can quickly respond to danger signals and attack and damage target cells by multiple mechanisms, including sudden release of reactive oxygen species (oxidative burst). C3a and C5a, anaphylatoxins released from activated C3 and C5 during complement activation, can recruit and activate neutrophils, with C5a being about 50-100 times more potent than C3a in activating neutrophils. Ehrengruber et al., FEBS letters. 1994; 346:181-4. Consistent with these previous reports, the inventors found that neutrophils were activated after MSC infusion in vivo, and, by using a C3aR antagonist, a C5aR antagonist, and purified C5a, demonstrated that C5a generated from MSC-initiated complement activation stimulated an oxygen burst by neutrophils that acted synergistically with MACs to damage MSCs.

CFH is the most potent complement regulator in serum, and impaired CFH function as a result of either certain gene polymorphisms or the development of anti-CFH autoantibodies leads to many diseases, including age-related macular degeneration and atypical hemolytic uremic syndrome due to excessive complement activation. M K L, Atkinson J P., J Intern Med. 2015; 277:294-305. Despite the facts that MSCs express all known intrinsic cell surface complement inhibitors and secrete factor H themselves (Tu et al., Stem cells and development. 2010; 19:1803-9), and that factor H exists at relatively high concentrations in the blood, immediately after MSC contact with the blood, MSC-activated complement overwhelms all these protective mechanisms to form MACs on MSCs and release C5a to mobilize neutrophils to damage the injected MSCs. These data further demonstrate the importance of local complement inhibition on MSCs in improving the treatment efficacy of MSCs. In addition, because, after MSC administration, complement activation primarily occurs on the MSCs, CFH in the blood cannot efficiently control activated complement on MSCs, making the painting of CFH onto MSCs even more critical for protecting the cells from complement-mediated direct and indirect attack. Although CFH is recognized as a complement alternative pathway inhibitor in the fluid phase, and the inventors previously showed that all three complement activation pathways are involved in damaging MSCs, because the alternative pathway serves as an amplification mechanism for all complement activation pathways and CFH also has co-factor activity for factor I to convert C3b into iC3b, which inhibits further complement activation from all three complement activation pathways, thus, painted CFH on MSCs should inhibit complement activation initiated from all three activation pathways, leading to robust protection of the MSCs from complement-mediated direct and indirect attack, as demonstrated in the inventors' in vitro and in vivo studies. At a concentration of 0.2-0.5 mg/ml in normal human plasma, purified CFH can be prepared on an industrial scale at a reasonable cost. Moreover, contrasting with other complement components in the blood, which are readily deactivated, CFH is stable even after incubation at 95° C., exposure to extreme pH, and storage at room temperature for several months, making the purification process relatively easy. These unique characteristics make the CFH-painting approach a practical method for improving current MSC-based therapies. The inventors envision that current MSCs under development could be further amended by the simple CFH “painting” method before infusion, and this approach should improve MSC survival and function in patients, thereby could help to successfully demonstrate the potential of MSCs in treating different diseases.

In summary, the inventors found that after administration, MSCs activate complement of the innate immune system. Activated complement leads to MAC assembly on MSCs to directly damage the cells, and it also produces C5a to stimulate neutrophils to further attack the administrated MSCs, resulting in impaired MSC survival and function. CFH, a potent native complement inhibitor can be activated and covalently painted onto MSCs to locally inhibit complement activation on MSCs, leading to significantly improved MSC viability and function after contact with complement and neutrophils both in vitro and in vivo. The inventors' results suggest that painting CFH onto MSCs before the administration could be a simple and effective approach to improve the outcome of current MSC-based therapies.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

What is claimed is:

1. A protected cell, comprising an immunogenic animal cell coated with a complement inhibitor.

2. The protected cell of claim 1, wherein the immunogenic animal cell is an autologous or allogenic stem cell.

3. The protected cell of claim 2, wherein the allogenic stem cell is a mesenchymal stem cell.

4. The protected cell of claim 1, wherein the complement inhibitor is heparin.

5. The protected cell of claim 1, wherein the complement inhibitor is factor H.

6. The protected cell of claim 1, wherein the complement inhibitor is covalently linked to the immunogenic animal cell.

7. The protected cell of claim 1, wherein the immunogenic animal cell is an autologous or allogenic mesenchymal stem cell, and the complement inhibitor is heparin or factor H.

8. A method of increasing immunogenic cell viability, comprising administering an immunogenic animal cell and a complement inhibitor to a subject.

9. The method of claim 8, wherein the immunogenic animal cell is coated with the complement inhibitor.

10. The method of claim 9, wherein the immunogenic animal cell is an autologous or allogenic stem cell.

11. The method of claim 10, wherein the allogenic stem cell is a mesenchymal stem cell.

12. The method of claim 9, wherein the complement inhibitor is heparin.

13. The method of claim 9, wherein the complement inhibitor is factor H.

14. The method of claim 9, wherein the complement inhibitor is covalently linked to the immunogenic animal cell.

15. The method of claim 8, wherein the immunogenic animal cell is an autologous or allogenic mesenchymal stem cell, and the complement inhibitor is heparin or factor H.

16. The method of claim 8, wherein the subject is a human in need of cell transplantation.

17. The method of claim 10, wherein the subject is a human in need of autologous or allogenic stem cell transplantation.

18. The method of claim 8, wherein the immunogenic animal cells are viable for at least twice as long as corresponding immunogenic animal cells administered to a subject without a complement inhibitor.
